(19) United States
(12) Patent Application Publication
(10) Pub. No.: US 2025/0082833 A1
(43) Pub. Date: Mar. 13, 2025

(54) MEASUREMENT OF RECIRCULATION BY MEANS OF TWO INTERIM SWITCHES HAVING KINETICALLY DIFFERENT DIFFUSION STATES

(71) Applicant: B. Braun Avitum AG, Melsungen (DE)

(72) Inventor: Waldemar Janik, Melsungen (DE)

(21) Appl. No.: 18/572,414

(22) PCT Filed: Jun. 24, 2022
(86) PCT No.: PCT/EP2022/067353

§ 371 Date: Dec. 20, 2023

(30) Foreign Application Priority Data
Jun. 24, 2021 (DE) 10 2021 116 343.9

(51) Int. Cl. A61M 1/36

(52) U.S. Cl. CPC A61M 1/3621; A61M 1/3607

(57) ABSTRACT

A blood treatment machine, module device and recirculation determination method feature a dialyser. A sensor is connected downstream of the dialyser and connected to a control unit that determines recirculation without blood-side bolus administration. The control unit: acquires the signal of a variable of consumed dialysis liquid measured by the sensor; switches the machine from a base mode into an interim mode, in which a dialysis liquid amount is confined in the dialyser on the dialysis liquid membrane side while blood flows in the blood circuit; changes into the base mode to supply the previously confined dialysis liquid amount as a first or second dialysis liquid bolus to the sensor to measure a signal change relative to a base signal as a corresponding first or second bolus signal; and deduces a recirculation from a deviation of the second bolus signal compared to the first bolus signal.

### CROSS-REFERENCE TO RELATED APPLICATIONS

This application is the United States national stage entry of International Application No. PCT/EP2022/067353, filed on Jun. 24, 2022, and claims priority to German Application No. 10 2021 116 343.9, filed on Jun. 24, 2021. The contents of International Application No. PCT/EP2022/067353 and German Application No. 10 2021 116 343.9 are incorporated by reference herein in their entireties.

### FIELD

The present disclosure relates to an extracorporeal blood treatment machine, especially dialysis machine, as a device for recirculation measurement in an extracorporeal blood treatment, for example hemodialysis, hemofiltration, and/or hemodiafiltration, and a module device for recirculation measurement configured for retrofitting to an extracorporeal blood treatment machine. Furthermore, a corresponding method for recirculation measurement is proposed.

### BACKGROUND

In an extracorporeal blood treatment, for example, a blood purification in the form of hemodialysis, hemofiltration, or hemodiafiltration, blood is taken from a dialysis patient via an arterial vascular access, treated in a dialyser, and subsequently returned to the patient via a venous vascular access. The patient's blood is thus passed through an extracorporeal circuit. In chronically ill patients, extracorporeal blood treatments are performed so frequently that the vein through which the blood is returned after treatment would become inflamed and sticky in the long run. For this reason, such patients are surgically fitted with a so-called shunt as access to the vascular system, which is a cross link between the artery and the vein of the patient and is used as a permanent puncture site. As a result of the shunt, the vascular wall of the vein thickens, so that it is easier to puncture and hence enables easier access for dialysis. In most cases such shunt is integrated in the arm of a patient.

Through the cross link between the artery and the vein, however, a blood exchange of venous and arterial blood also takes place, especially if, during the blood treatment, the blood flow at the blood pump is set too high and consequently the blood to be returned into the patient's shunt partially passes over to the arterial vascular access of the patient. Consequently, the already purified venous blood dilutes the still unpurified arterial blood, so that an impairment of the blood treatment efficiency or, respectively, the effectiveness of the blood treatment occurs. From the point of view of a (transmembrane) mass transport or, respectively, the diffusion between, on the one hand, a blood (membrane) side and, on the other hand, a dialysate (membrane) side (or, respectively, dialysis liquid-side) (of a dialysis membrane) of a dialyser, this is based on a disadvantageously reduced concentration gradient or, respectively, a disadvantageously reduced driving potential. As a result, the treatment time is adversely prolonged.

Such a process, in which the blood that is already purified and returned into the patient flows from the patient's vein via the vascular access, e.g. shunt, into the artery and from there enters into the extracorporeal blood circuit again, is called recirculation or, respectively, recirculation fraction, recirculation value (in %) and may be determined qualitatively and quantitatively by different methods. In other words, the recirculation refers to the (volume flow or, respectively, flow rate) ratio of the already purified blood sucked back from the venous access into the arterial access (quasi repeatedly) in the shunt, in relation to the current (total) blood flow. Thereby, the (total) blood flow (volume flow or, respectively, the blood flow rate) in the extracorporeal blood tubing system is known, as it is adjustable at the machine (as the delivery rate of the blood pump). A quantitative recirculation measurement is employed, among other things, for monitoring of the shunt condition and for checking of the blood treatment settings, such as the delivery rate of the blood pump.

From the prior art several different methods for determining of the recirculation fraction are known.

A number of frequently used methods for recirculation determination are based on the principle of a blood-side bolus administration. For example, according to this principle, the prior art knows the generation of a defined temperature bolus, which is applied in the venous blood branch, with subsequent temperature measurement in the arterial branch, so that based on the applied temperature difference and the measured temperature difference, a conclusion about the recirculation rate can be mathematically formed. It is also known to measure another indicator instead of the temperature, for example a specific substance concentration or the conductivity, whereby the indicator has previously been applied in the form of an (indicator) bolus. Also, the bolus may be applied dialysis liquid-side upstream of the dialyser and a corresponding measurement may be performed dialysis liquid-side downstream of the dialyser, from which the recirculation is determinable.

For example, from the patent document DE 197 02 441 C1 a device and a method for determining the recirculation in an extracorporeal blood treatment using a shunt is known, whereby an indicator variable, for example a concentration bolus, is generated in the dialysis liquid circuit upstream of the dialyser and the concentration is observed over a defined time period later in the dialysis liquid circuit downstream of the dialyser in order to draw a conclusion about the recirculation.

Also in EP 2 783 715 A1 a method for recirculation measurement is disclosed, in which a recirculation is determinable by blood-side bolus administration and dialysis liquid-side spectrophotometric measurement.

As a further example, the patent application U.S. Pat. No. 5,588,959 A describes a device and method for a recirculation measurement by temperature. Thereby, the blood is cooled at the venous tube section and the temperature of the blood is measured in the arterial tube section.

Furthermore, the patent application WO 96/08305 A1 discloses a method for recirculation determination in extracorporeal blood treatments, in which an indicator is added in the venous blood branch and a measured value associated with the indicator is acquired by means of a detector at the arterial blood branch and a recirculation rate is calculated on the basis of the dilution curve.

As an alternative to the prior art cited above according to the principle of the blood-side bolus administration, DE 10 2013 103 221 A1 discloses a recirculation measurement according to a transient method after changing of the extracorporeal blood flow. In the mathematical determination of the recirculation according to the transient method after changing of the extracorporeal blood flow a damping parameter with regard to the transient process with and without recirculation is included, for which the variable concentration of a substance at the dialysate outlet c_{DO} is acquired by using an optical sensor at the dialysate outlet. Thereby, the recirculation determination is based on a calculation model with the following equation or, respectively, a corresponding transformation (using a known equation from the fundamentals of Michaels [see introductory part of WO 2020/212332 A1 citing DE 10 2013 103 221 A1]):

$$R = \frac{1 - \frac{{c}_{DO} \cdot {Q}_{D}}{{c}_{sys} \cdot {K}_{D}}}{1 - \frac{{c}_{DO} \cdot {Q}_{D}}{{c}_{sys} \cdot {K}_{D}} + \frac{{c}_{DO} \cdot {Q}_{D}}{{c}_{sys} \cdot {Q}_{B}}}$$

Thereby, the following applies:

| | | |
|---|---|---|
| R | recirculation value or, | |
| | respectively, recirculation rate | |
| | (0 . . . 1 or, respectively, 0 . . . 100%) | |
| c_{DO} | concentration of a substance at | measured value (current) and/or |
| | the dialysate outlet | measurable |
| c_{sys} | c_sys systemic blood component | measured value/peak value |
| | concentration which is not | |
| | recirculation-affected | |
| | (concentration in the patient) | |
| Q_{D} | dialysis fluid flow rate | known or, respectively, adjustable |
| Q_{B} | Blutflussrate im extrakorporalen | known or, respectively, adjustable |
| | Blutschlauchsystem | (cf. DE 10 2013 103 221 A1, |
| | | para. [0123]) |
| K_{D} | theoretical clearance of the | function of (K_{0A}, Q_{B}, Q_{D}) → thus known |
| | dialyser | |
| K_{0A} | dialyser-specific clearance | known (lookup table), i.e. specific |
| | coefficient | clearance coefficient which is dependent |
| | | on the currently used dialyser and dialysis |
| | | fluid used and which can be determined |
| | | (empirically) in advance |

It is of disadvantage here that, for determination of c_{sys} before the blood treatment, first a blood sample from the patient must be taken and c_{sys} be measured (in the analytical laboratory). Then, the recirculation R during the blood treatment can be calculated using the above equation.

The aforementioned prior art always has however the disadvantage that either an increased apparative expenditure is required, for example by providing of temperature control devices or devices for a substance or, respectively, concentration bolus administration, or a separate blood value determination must be carried out before the treatment. Furthermore, the determination of c_{sys} from a patient blood sample is laborious and therefore not satisfactory.

WO 2020/212 332 A1 of the present applicant, regarded as closest prior art, which is hereby expressly made part of the present disclosure by reference, already overcomes the above-mentioned disadvantages and discloses a generic extracorporeal blood treatment machine. In this respect, the disclosure of WO 2020/212 332 A1 teaches a device for extracorporeal blood treatment, which is configured to determine the systemic blood component concentration c_{sys} (concentration of a blood component in the blood of the patient's body) during the blood treatment and especially exclusively with the equipment provided in principle at a device for extracorporeal blood treatment and to deduce the recirculation R then on the basis of the above known equation.

### SUMMARY

It is thus the object of the disclosure to find a technical solution alternative to the disclosure of WO 2020/212 332 A1, in order to overcome or at least mitigate the disadvantages of the prior art according to the principle of the blood-side bolus administration or according to the transient method and especially to provide a device for recirculation measurement in an extracorporeal blood treatment, for example using a shunt, which is feasible without additional apparative effort, for example a temperature control device, and/or procedural or, respectively, clinical effort, such as, for example, a separate blood sample collection before the treatment.

Compared to the disclosure of WO 2020/212 332 A1, there is the additional object to provide a particularly cost-effective and easy-to-perform recirculation measurement, which is not dependent on the systemic blood fraction concentration c_{sys} and the other machine or, respectively, therapy parameters further entering into the determination.

According to the disclosure, an extracorporeal blood treatment machine, especially a dialysis machine, has a dialyser and at least one sensor device, which is connected on a dialysis liquid-side (or, respectively, in the dialysis liquid circuit) downstream of the dialyser and electrically connected to a control and computing unit. The term of the dialyser refers to a device for blood purification, such as hemodialysis, hemofiltration and/or hemodiafiltration, especially a dialysis module, further preferably a hollow fiber module. Thereby, according to the disclosure, the control and computing unit for determination of a presence of a recirculation without blood-side bolus administration is provided and designed (or, respectively, configured) to acquire the at least one signal, herein referred to as S_{DO}, of a physical and/or chemical variable of the consumed dialysis liquid, measured by the at least one sensor device for quantitative determination of at least one preferably selected or selectable blood component in the consumed dialysis liquid, in its course over time [i.e., as a time-variable (measurement) signal or, respectively, as a measurement curve, especially as a functional dependence S_{DO}=ƒ(t)]. Thereby, the at least one sensor device may acquire the measurement curve or, respectively, the temporal course by means of continuous and/or discontinuous, e.g. clocked, measurement.

Further, the control and computing unit is thereby, according to the disclosure, configured to operate the extracorporeal blood treatment machine in a base mode for continuous operation, in which the dialysis liquid flow through the dialyser is admitted; and to measure the signal corresponding to the dialysis liquid consumed in the base mode as a base signal (or, respectively, as a baseline, a reference value signal, a normalization value).

Thereby, the term (of the) base mode refers to the continuous operation in which the (continuous) dialysis liquid flow through the dialyser is admitted. Thereby especially, the dialysis liquid rate may be essentially constant, so that the flow in the dialyser corresponds to a steady state.

Especially, the term (of the) base mode means the (normal) dialysis operation mode provided for the core function of the dialysis or, respectively, of the extracorporeal blood treatment. The base signal is referred to herein as S_{DO, pre}, whereby the suffix “pre” is chosen to denote a state preceding a change of operation mode in a merely nominal and non-limiting manner. Due to the characteristic of a base signal in the base mode as the (normal) dialysis operation mode, the skilled person understands that the base signal equally occurs or, respectively, may be acquired in a subsequent state or, respectively, at a later point of time (suffix “post”).

Further, the control and computing unit is thereby, according to the disclosure, configured to switch the extracorporeal blood treatment machine from the base mode into an interim mode for (the) interim operation, in which a dialysis liquid amount is confined in the dialyser on the dialysis liquid membrane side, while the blood flow in the extracorporeal blood circuit is operated at a set blood flow rate, whereby the blood on the blood membrane side (8B) continues to flow, for the respective duration of a pre-determined and/or predeterminable interim time interval. Especially the interim mode may relate to a bypass mode (or, respectively, an operation mode in a bypass circuit or, respectively, side connection circuit).

Subsequent to the preceding foregoing switching step into the interim mode, especially bypass mode, the control and computing unit, according to the disclosure, is configured to change into the base mode in order to supply the previously confined dialysis liquid amount as a respective dialysis liquid bolus to the at least one sensor device. This, according to the disclosure, serves to measure a signal change (or, respectively, signal deviation), corresponding to the dialysis liquid bolus, relative to the (or, respectively, in relation to the, corrected by the) base signal as a corresponding bolus signal. In other words, the dialysis liquid bolus, by the switching back into the base mode, is pumped past and passes the at least one sensor device with the resuming dialysis liquid rate.

Depending on the preferred alternative and/or cumulative embodiments further disclosed in detail below, the corresponding bolus signal may be: i) acquired as a (maximum or minimum or sign-neutral) peak signal or, respectively, an extremum, herein referred to as S_{DO, ext}; and/or ii) acquired as an integral total area or, respectively, a total area integral, herein referred to as A; and/or iii) acquired as an integral partial area or, respectively, partial area integral, herein referred to as Aₚₐᵣₜ.

Further, the control and computing unit is thereby, according to the disclosure, configured to switch the interim mode for a first interim time interval for establishment of equilibrium of the mass transfer. For this purpose, a first dialysis liquid amount in the dialyser is confined at least until a concentration equilibrium of the blood component is established on the dialysis liquid membrane side and the blood membrane side of the dialyser, namely for the duration of the first interim time interval, which equilibrium is no longer changing or is only still changing to an unsubstantial degree.

Further, the control and computing unit is thereby, according to the disclosure, configured for forming a measurement cycle to switch to the interim mode for a second interim time interval (which is) shorter than the first interim time interval. For this purpose, a second dialysis liquid amount in the dialyser is confined only as long as the concentration equilibrium of the blood component is not yet established (or, respectively, has been reached), namely for the duration of the second interim time interval.

Further, the control and computing unit is thereby, according to the disclosure, configured to deduce, from a deviation of a second bolus signal as result of the second interim time interval compared to a first bolus signal as result of the first interim time interval, the presence of the recirculation.

In summary, it is an essential idea of the present disclosure to perform successively (in any order) at least two (separate, independent) interim modes or, respectively, circuits, especially bypass modes (also referred to as side connection circuits), of different duration or, respectively, for interim time intervals of different length. Thereby, the interim time intervals of different duration mark qualitatively significantly different kinetic states, namely on the one hand an equilibrium state, on the other hand a state of disequilibrium (or, respectively, a state in advance to reaching the equilibrium state). The two respective signal deflections, which can be determined at the dialysate outlet according to the two interim modes or, respectively, circuits, especially bypass modes, are set in relation (to each other). From this relation, the recirculation is, according to the disclosure, determined qualitatively (i.e. as such) or, respectively, the (especially significant or, respectively, relevant) presence of the recirculation is detected. In preferred alternative and/or cumulative embodiments, further disclosed in detail below, the recirculation may furthermore be quantitatively (i.e. as recirculation value, especially as recirculation fraction in %) determined or, respectively, calculated.

According to the present disclosure, there are several advantages or, respectively, improvements compared to the prior art. In this respect, a particularly simple and fast, yet at the same time sensitive recirculation measurement is proposed. As an automatic recirculation measurement, this takes place inconspicuously and for the patient not disturbingly in the background. Thus, the present technical solution for detection or, respectively, determination of a, if applicable, present recirculation in the simplest case does not require any knowledge of further relevant variables or, respectively, adjustment parameters. Especially, no change and/or adjustment, calibration etc. of other setting parameters is required specifically for the purpose of the recirculation determination, such as of the dialysis liquid and/or the blood flow rate. Therefore, the present disclosure is not only particularly suitable for relieving clinic staff, but especially also for dialysis applications by the patient alone at home. Especially the disclosure offers a technical solution for which no infusion solutions are necessary.

Due to the fact that the signals are directly processed by the control and computing unit, it is a particularly robust detection or, respectively, measurement method that is not susceptible to noise. Not to be neglected is further the cost advantage regarding components and maintenance. In this respect, the disclosure can be performed with sensor technology established in dialysis machines, so that no additional equipment is required.

For this purpose, the disclosure makes use of at least one sensor device at or downstream to a dialysis liquid-side dialyser outlet, especially measuring the conductivity, cumulatively or alternatively further preferably optically operating, which are generally used for detection/determination of specific blood components such as uremic toxins (e.g. urea) in the consumed dialysis liquid, and of an electronic control or, respectively, control and computing unit, preferably on-board the dialysis machine.

Thereby, the electronic control, according to the disclosure, is provided and adapted to switch the dialysis machine for execution of a measurement cycle at least twice, respectively, into an interim mode, especially into a bypass mode or, respectively, into a side connection (circuit) mode, in which dialysis liquid in the dialyser is confined on the one hand for a first interim time interval and on the other hand for a second interim time interval.

With regard to the interim mode, it is irrelevant in which order the first interim time interval and the second interim time interval are (temporarily) switched into from the base mode prevailing before, in between and/or after. In other words, according to the disclosure, it does not matter whether—in each case starting from the base mode and returning to the base mode after the lapse of time—switching takes place first into the first interim time interval and later into the second interim time interval is switched; or first into the second interim time interval and later into the first interim time interval. In this respect, the interfacial physical processes of the mass transport or, respectively, the (transmembrane) diffusion of at least one blood component, transiting from the blood into the respective dialysis liquid bolus (or, respectively, into the temporarily stationarily residencing, i.e. confined delta volume of the dialysis liquid membrane side) as well as the kinetics of the interfacial physical processes occur independently of the path. In other words, the interfacial physical processes during the first interim time interval with respect to the first dialysis liquid bolus do not influence the processes during the second interim time interval with respect to the second dialysis liquid bolus; and vice versa.

Merely, the first dialysis liquid bolus, corresponding to the (long) first interim time interval, represents an equilibrium state that has been reached. For this purpose, the (long) first interim time interval is determined or, respectively, selected in advance such that the first dialysis liquid bolus is confined (at least) until a blood component concentration equilibrium is established/has been reached on the dialysis liquid membrane side and the blood membrane side of the dialyser that no longer changes (or only to an unsubstantial degree). I.e., during the confinement phase, the purification of the blood in the dialyser decreases and approaches zero, which is why, after a sufficiently long residence time, a diffusion equilibrium finally prevails between the blood and dialysis liquid membrane sides. This (balance) amount or, respectively, a (balance) volume of consumed dialysis liquid temporarily confined in the dialyser, referred to as dialysis liquid bolus, thus has a blood component concentration essentially corresponding to the patient's blood.

In the context of the first interim time interval, it should be noted that the kinetics of the mass transport or, respectively, of the diffusion and thus a respective point of time at which a respective diffusion equilibrium is reached depends not only on the specific blood component, for example a specific uremic toxin, if applicable, but, if applicable, also on the efficiency of the blood treatment, i.e. on the actually present recirculation. If the recirculation is lowly pronounced or even absent, the diffusion equilibrium, if applicable, is reached faster than if the recirculation is high, because recirculated blood leads to a dilution of the arterial blood to be purified and thus impairs the efficiency of the blood purification. The duration until reaching of the diffusion equilibrium between the blood and the dialysis liquid membrane side is significant but dependent on the set blood flow rate and the dialyser size. The higher the blood flow rate and the smaller the dialyser size, the faster diffusive equilibrium is reached.

Now, in order to ensure that the duration of the interim mode, especially the bypass mode, actually corresponds to at least the duration until which the diffusion equilibrium of a specific blood component has been established in the dialyser, the first interim time interval must always be designed for the maximum duration of the diffusion equilibrium setting. This is provided to the control and computing unit in advance, either by retrieving the information from a database/data set or by input from a user, and may be determinable depending on the patient, his shunt condition and/or further specific empirical values, such as from an empirical determination in the laboratory or evaluation of past blood treatments.

In contrast, or, respectively, relative to the first interim time interval, the (short) second interim time interval is, in advance, determined or, respectively, selected to be shortened (by an absolute and/or proportional shortening value) such that the second dialysis liquid bolus is confined (maximally) for such a long time that, on the dialysis liquid membrane side and the blood membrane side of the dialyser, blood component concentration equilibrium is/has been just not established or, respectively, in no case (any) that is not changing any more (or only insignificantly). In other words, the second dialysis liquid bolus represents, corresponding to the (short) second interim time interval, a kinetic state with (sufficient) distance in advance of an established equilibrium state, i.e. a state of disequilibrium. In other words, a still further running kinetics of the interfacial physical processes, which is determined by the presence of driving gradients or, respectively, concentration potentials, is terminated at the time of the end of the second interim time interval or, respectively, the prevailing state is, so to speak, ‘frozen’ for the subsequent measurement by the at least one sensor device downstream.

The duration to be expected for the first (or, respectively, in relation thereto: second) interim time interval is further dependent on what kind of dialyser is used, for example on the condition (new or reused) and/or the (available) filter area and/or the (dialysate-side) filling volume, and with which blood flow rate the blood to be cleaned is passed through the dialyser. Therefore, a data set is provided to the control and computing unit, from which the target duration(s) for the first (or, respectively, second) interim time interval and the target blood flow rate to be set can be deduced, considering the maximum possible recirculation and the characteristics of the specifically used/connected dialyser. Alternatively, it is also conceivable to deduce the target duration(s) for the first (or, respectively, second) interim time interval also as a function of a pre-determined blood flow rate.

With the switching over from the interim mode, especially bypass mode, (again back) into the base mode, the flow-through of the dialyser occurs again, so that the previously temporarily confined (balance) amount is then released to be supplied like a dialysis liquid bolus to the at least one sensor device downstream of the dialysis liquid outlet. Thereafter, the at least one sensor device measures or, respectively, acquires a chemical and/or physical variable correlating with its blood component concentration as a bolus signal (corresponding to the respective dialysis liquid bolus). According to the disclosure, the bolus signal is noticeable or, respectively, acquired as a change (or, respectively, a deviation, a deflection) in the signal (or, respectively, measurement signal, sensor signal) of the at least one sensor device (immediately) after the release of the dialysis liquid bolus from the dialyser. Thereby, it is irrelevant whether the bolus signal deflects in the positive or negative ordinate axis.

At this point, it should be noted that the term “confined in the dialyser” specifically is to be construed as a confinement of the dialysis liquid on a dialysis liquid membrane side of the dialyser. The term “confined” is further to be understood as not or, respectively, essentially not flowing through. Further, the confined space may, in some circumstances, still comprise portions of the dialysis liquid inlet/outlet line. For example, if one were to assume that, e.g., by activating a dialyser bypass with a significantly lower flow resistance (compared with the dialyser), a flow of the dialysis liquid through the dialyser is interrupted or, respectively, essentially interrupted, the tightly closing (blocking) valves for such a bypass circuit could be dispensed with. In this case, the liquid would not really be ‘confined’ but the flow-through of dialysis liquid through the dialyser would be reduced to near zero.

In other words, in the bypass mode as the preferred interim mode, the dialysis liquid membrane side in the dialyser is not supplied with fresh dialysis liquid or, respectively, the filter membrane dialysis is not flushed liquid-side with fresh dialysis liquid. Rather, a (first or, respectively, second) dialysis liquid amount present on the dialysis liquid membrane side is kept there, preferably using (check) valves which are arranged in the dialysis liquid inlet line and the dialysis liquid outlet line. Instead of (locking) valves, other elements suitable for fluid shut-off, such as pumps, are of course also conceivable. Now, while the dialysis liquid on the dialysis liquid membrane side is kept there, the blood flow in the extracorporeal blood circuit is operated at a set blood flow rate. The blood membrane side of the dialyser is thus further supplied with arterial blood or, respectively, blood to be purified or, respectively, the filter membrane on the blood side is further flushed with arterial blood or, respectively, blood to be purified. Consequently, as is usual in dialysis treatments, a transfer of substances dissolved in the blood (blood components) through the filter membrane into the dialysis liquid takes place due to diffusion, that is,—in the absence of interruption—until there is no longer a concentration gradient between the blood present on the blood membrane side and the dialysis liquid present on the dialysis liquid-side, i.e., in other words, a diffusion equilibrium is reached. On the basis of this kinetic point of time, the first interim time interval is definable. Since the blood on the blood membrane side of the dialyser continues to flow while the dialysis liquid is stationary on the dialysis liquid membrane side of the dialyser, blood components continue to accumulate in the stationary dialysis liquid until these have reached at least essentially the same concentration in the stationary dialysis liquid as in the blood flowing further through the dialyser. In other words, at the point of time when the diffusion equilibrium of a blood component between the stationary dialysis liquid and the flowing blood is (essentially) reached, the concentration of the blood component dissolved in the stationary dialysis liquid corresponds at least essentially to the concentration of the blood component dissolved in the blood of the patient's body. At this point it should be noted that the interfacial physical processes determining variable “concentration” may also be represented by another parameter related to it, such as an electrical conductivity measurable by a sensor device and/or an absorbance.

Furthermore, with regard to the term of the interim mode, it is possible, further or, respectively, alternatively, to switch into the so-called sequential ultrafiltration instead of the bypass mode. Therein, a check valve downstream of the dialyser remains opened, so that a convective purification of the blood using an ultrafiltration device or, respectively, the dialyser is still provided during this time period. The ultrafiltration rate may be increased, decreased or kept constant in comparison to the ultrafiltration rate in the base mode or, respectively, main connection. In other words, the term of the interim mode is intended to comprise, in the manner of a generic term, the term of the bypass mode or, respectively, circuits in form of the bypass equally as the term of the sequential phase in the performance of sequential ultrafiltration. Since the disclosure is otherwise identical, for avoidance of repetition the preferred embodiments are explained in the following only on the basis of the case of the bypass circuits. Thereby, all alternative and/or cumulative technical features are intended to be equally disclosed also in connection with the sequential ultrafiltration.

If a recirculation is present, the blood entering into the dialyser during the (“short”) second interim time interval, i.e. during this time in advance of the establishment of equilibrium, is influenced by the venous blood flowing back and thus recirculation-affected. Consequently, during the (“short”) second interim time interval, the confined second dialysis liquid amount of the dialysis liquid membrane side, at (or, respectively, in the case of) the presence of recirculation, is recirculation-affected.

In contrast, during the (“long”) first interim time interval, as described above, the confined first dialysis liquid amount of the dialysis liquid membrane side adapts completely to the blood that is being continuously pumped through the dialyser. Since the composition of the arterial blood is no longer influenced by the dialysis liquid, the venous blood flowing back does not lead to any further change in the arterial blood, even in the presence of a recirculation (i.e., in the case of a recirculation being present, despite this).

Therefore, after a sufficiently long time in the bypass mode or, respectively, at the latest at the end of the first interim time interval, the blood in the arterial line is no longer recirculation-affected.

Consequently, the first dialysis liquid amount of the dialysis liquid membrane side that is enclosed or, respectively, confined during the (“long”) first interim time interval is not recirculation-affected, even in (or, respectively, in case of the) presence of a recirculation. In other words, it plays no (or, respectively, a negligible) role whether (a) recirculation is present or just not. Recirculation only plays a role insofar as the time span until (a) diffusion equilibrium is reached is changed (extended).

Preferably, alternatively or cumulatively, the first bolus signal and the second bolus signal may respectively correspond to a corresponding (in case of positive signal: maximum; or, respectively, in case of negative signal: minimum; or, respectively, sign-neutral) peak (or, respectively, extremum) in the signal of the at least one (especially first and/or second) sensor device. Thereby, the peak or, respectively, the extremum thereof is acquired immediately after the release of the respective first or, respectively, second dialysis liquid bolus from the dialyser. The (sign-neutral) maximum peak of the first bolus signal is referred to as S_{DO, ext, L}; the (sign-neutral) maximum peak of the second bolus signal is referred to as S_{DO, ext, K}.

Thereby, the control and computing unit is further provided and configured to, respectively, calculate a second difference, denoted D_{K}, from the maximum peak of the second bolus signal S_{DO, ext, K} and the base signal S_{DO, pre} as well as a first difference, denoted D_{L}, from the maximum peak of the first bolus signal S_{DO, ext, L} and the base signal S_{DO, pre}. In other words, the first and the second difference represent a delta value or, respectively, a signal value adjusted or, respectively, reduced by the base signal. Thus, the first and second differences represent the actual response or, respectively, signal response of the at least one sensor unit to the passage of the first or, respectively, second dialysis liquid bolus.

The respective differences between the respective peak or, respectively, extremum and the corresponding value before the interim mode, especially bypass mode, are described by the following two equations:

$${D}_{L} = {S}_{DO , ext , L} - {S}_{DO , pre , L}$$

$${D}_{K} = {S}_{DO , ext , K} - {S}_{DO , pre , K}$$

The value S_{DO,pre,L} corresponds to the steady state value, i.e. the base signal, shortly before the beginning or end of the first interim time interval, especially before the beginning or end of the longer lasting bypass circuit. It may also correspond to the steady state value shortly before the beginning or end of the second interim time interval, especially before the beginning or end of the shorter lasting bypass circuit, denoted as S_{DO, pre, K}, or vice versa.

Thereby, the control and computing unit is further provided and designed to calculate or, respectively, deduce a delta ratio (as ratio value) from the second difference D_{K} in relation to the first difference D_{L}. Further, the control and computing unit is thereby provided and designed to determine a current recirculation value R (in %), based on the deduced delta ratio, referred to as V_{D}, and a predefined calculation model.

The ratio of the second difference to the first difference is referred to as the delta ratio V_{D}:

$${V}_{D} = \frac{{D}_{K}}{{D}_{L}}$$

Thus, the current recirculation value R can be determined as function of this relation or, respectively, of the delta ratio V_{D}:

R=ƒ(V_{D})

By the term calculation model the skilled person understands especially the plurality of numerical-mathematical methods known to him, in order to represent a mathematical or, respectively, functional relation or, respectively, a correlation between parameters. The relation may preferably be provided as a pre-determined or, respectively, stored lookup table, characteristic curve, characteristic diagram and/or the like. For example, the relation may have been deduced in advance by means of multiple regression and/or by means of an artificial neural network and/or any other machine learning method. Especially it may be a, preferably multi-parametric or, respectively, multi-dimensional, characteristic diagram, which is provided in form of fitted characteristic curves or, respectively, model function equations mapped or, respectively, broken down (on a storage unit). For example, in engineering, fitting and/or regression methods are known in many ways to transform an e.g. experimentally deduced (graphical) characteristic diagram into a formulaic (numerical) notation, e.g. as polynomial. Thereby, a respective single parameter of the calculation model may correspond to a genuine or, respectively, real physical and/or chemical variable, e.g. a concentration, or to a (natural) constant or to a coefficient of the dialyser or, respectively, of the dialysis machine; and/or a respective single parameter of the calculation model may just not correspond to a real parameter in the scientific sense, but represent a so-called fitting parameter and/or compound parameter. Further it is predescribed that a pre-determined characteristic diagram is valid only in an application interval.

Alternatively for evaluation of the signal differences also respective total areas below the two signal deflections A_{K} and A_{L} and/or respective partial areas below the two signal deflections A_{part, K} and A_{part, L} can be brought into relation:

$${V}_{A} = \frac{{A}_{K}}{{A}_{L}}$$

$$total area integral ratio$$

Or, respectively:

$${V}_{A , part} = \frac{{A}_{part , K}}{{A}_{part , L}}$$

$$partial area integral ratio$$

In this way, the recirculation can also be determined as a function of the area ratios in terms of the total area integral ratio or, respectively, partial area integral ratio:

$$R = f ( {V}_{A} )$$

$$R = f ( {V}_{A , part} )$$

Preferably, alternatively or cumulatively, the first bolus signal may also correspond to a (first) total area integral, denoted as A_{L}, in the signal of the at least one sensor device immediately after the release of the first dialysis liquid bolus from the dialyser and the second bolus signal (may correspond) to a (second) total area integral, denoted as A_{K}, in the signal of the at least one sensor device immediately after the release of the second dialysis liquid bolus from the dialyser. Thereby, the first total area integral A_{L} and the second total area integral A_{K} are each adjusted by the base signal S_{DO, pre}. As explained above, the base signal S_{DO, pre} may especially, but not limitingly, relate to a preceding one (as indicated by the suffix in the designation). In this respect, the base signal may equally be provided or, respectively, acquired as a subsequent base signal. Also, for all adjusted bolus signals or, respectively, ratios, it is conceivable to use the same base signal for both the first and the second bolus signal.

Thereby, the control and computing unit is further therefor provided and configured to calculate or, respectively, to deduce a total area integral ratio (as ratio value) from the second total area integral A_{K} in relation to the first total area integral AL. Further thereby, the control and computing unit is therefor provided and configured to determine, based on the deduced total area integral ratio, referred to as V_{A}, and a predefined calculation model, a current recirculation value R (in %).

Preferably, alternatively or cumulatively, the first bolus signal may also correspond to a (first) partial area integral, denoted as A_{part, L}, in the signal of the at least one sensor device immediately after the release of the first dialysis liquid bolus from the dialyser and the second bolus signal may correspond to a (second) partial area integral, denoted A_{part, K}, in the signal of the at least one sensor device immediately after the release of the second dialysis liquid bolus from the dialyser. Thereby, the first partial area integral A_{part, L} and the second partial area integral A_{part, K} are each adjusted by the base signal S_{DO, pre}. As explained above, this may especially, but not limitingly, relate to a preceding base signal S_{DO, pre}.

Thereby, the control and computing unit is further therefor provided and configured to calculate or, respectively, to deduce a partial area integral ratio (as ratio value) from the second partial area integral A_{part, K} in relation to the first partial area integral A_{part, L}. Further thereby, the control and computing unit is therefor provided and configured to determine, based on the deduced partial area integral ratio, referred to as V_{A, part}, and a predefined calculation model, a current recirculation value R (in %).

Especially, the first partial area integral A_{part, L} or, respectively, the second partial area integral A_{part, K} may thereby represent a proportional (by a respective proportion value) section or, respectively, a proportional area of the first total area integral A_{L} or, respectively, of the second total area integral A_{K}. Especially, it may be provided that the respective proportion value for the first partial area integral A_{part, L} and the second partial area integral A_{part, K} is selected identical. For example, the respective proportion value may be less than or equal to 90%, further preferably less than or equal to 80% and especially preferably less than or equal to 70%. Especially, it may be preferred to direct a proportional area of the first total area integral A_{L} or, respectively, of the second total area integral A_{K} to an initial partial area and/or a partial area centered around the corresponding peak or, respectively, to provide it as such, e.g. to cut off or, respectively, to ignore an extending drag of the first or, respectively, second total area integral for the evaluation. This causes the advantage of a faster and/or more precise measurement.

Preferably, alternatively or cumulatively, the calculation model for calculation of the current recirculation value may take into account a dialysis liquid flow rate of the dialysis liquid circuit, denoted as Q_{D}, and/or a blood flow rate, denoted as Q_{B}, in the extracorporeal blood tubing system of the blood circuit and/or a theoretical clearance of the dialyser. Especially, the calculation model may further take into account at least one (further parameter) out of a number of further machine, dialyser and/or setting parameters (especially regarding the blood treatment machine) as relevant variable. Especially, the at least one further parameter may relate to the blood treatment machine. These parameters may preferably be system-known to the extracorporeal blood treatment machine system and thus be used by the control and computing unit. From-case-to-case it may be preferred or (just) not preferred that the calculation model is designed to consider the influence of the dialyser type as negligible or, respectively, to (mathematically) neglect it. This may support a further simplification of the determination according to the disclosure in such a case, in which it has been verified by means of measurements that a consideration of the dialyser does not lead to an improvement of the accuracy of the determination of the presence of the recirculation or, respectively, of the current recirculation value. Overall, the optional consideration of at least one further parameter contributes advantageously to a further increase of the accuracy and/or the reproducibility of the determination of the presence of the recirculation or, respectively, of the current recirculation value.

Thus, it has been shown that the accuracy of the recirculation determination is increased if, in addition to the above disclosed ratios, i.e. the delta ratio V_{D}, the total area integral ratio V_{A} and/or the partial area integral ratio V_{A, part}, at least one or a plurality of further parameters is added and/or even several of the ratios are combined with each other.

The function for determination of the recirculation can be generalized as follows:

$$R = f ( {V}_{D} , {V}_{A} , {V}_{A , part} , {S}_{DO , ext , K} , {S}_{DO , ext , L} , {S}_{DO , pre , K} , {S}_{DO , pre , L} , {Q}_{b} , {Q}_{d} )$$

This relation may be provided as stored calculation model, especially as a lookup table or as a characteristic curve (diagram).

Thereby, the result of the recirculation determination may preferably be displayed to the user, especially the medical or, respectively, clinical staff and/or the patient, e.g. on a display device of the extracorporeal blood treatment device or on a display device which is in contact with the extracorporeal blood treatment device wirelessly or by wire. The recirculation may be output both qualitatively (presence of a recirculation as such) and as a quantitative measure or, respectively, as a current recirculation value (in %). Thereby, a qualitative output means especially that it is indicated whether a medically relevant recirculation (e.g. recirculation value greater than or equal to 15%) is present or not. A semi-quantitative output may relate to e.g. a traffic light color system.

The respective duration of the interim circuits, especially the duration of the bypass circuits (or, respectively, the side connection circuits), especially the optimal duration of the longer bypass circuit, i.e. the optimal first interim time interval, depends on the set and/or effective blood flow rate through the dialyser and on the dialyser (type) itself. Thus, it takes longer to reach complete saturation or, respectively, an (asymptotically arriving) establishment of equilibrium in one case with a low blood flow rate and a large dialyser than in another case with a higher blood flow rate and a smaller dialyser. This information can be used for optimizing the duration of the longer bypass circuit, i.e. the first interim time interval.

Preferably, alternatively or cumulatively, the duration of the first interim time interval may also be selected such that all combinations of blood flow rates and dialyser sizes pre-determined or, respectively, predefined as relevant are covered. Thus, preferably the first interim time interval for establishment of equilibrium of the mass transfer may be set and/or settable to a blanket value. Thereby, especially the first interim time interval may be set and/or settable to a (blanket) duration between 2 and 6 minutes, further preferably between 4 and 5.5 minutes, especially of circa 5 minutes. This serves to blanket-specify a duration for the first interim time interval which is sufficient with certainty for the establishment of equilibrium of the mass transfer and which on the other hand is optimized not to excessively prolong the measurement cycle or, respectively, the change from the base mode. A blanket, especially first, interim time interval has the advantage that for the extracorporeal blood treatment machine as well as the corresponding monitoring method it does not have to be known which dialyser is used.

Preferably, alternatively or cumulatively, the second interim time interval prior to the establishment of equilibrium of the mass transfer may be set and/or settable to a blanket value. Thereby, the second interim time interval may preferably be less than or equal to 1 minute, further preferably 10 to 20 seconds, especially circa 14 seconds. Thus, a particularly advantageous duration for the second interim time interval, which has been proven in experimental test series, is recommended or, respectively, specified in advance by the manufacturer. This results in the advantage of the greatest possible independence from other influencing variables and thus in a so-called robustness against possible disturbing influences on the measurement accuracy or, respectively, significance of the determination of the presence of the recirculation or, respectively, of the current recirculation value.

Preferably, alternatively or cumulatively, the shortening factor by which the second interim time interval is shortened in respect (or, respectively, in relation) to the first interim time interval may be greater than or equal to 1:5, further preferably greater than or equal to 1:8 and especially greater than or equal to 1:20.

Preferably, alternatively or cumulatively, the control and computing unit may be further configured to execute the measurement cycle automatically, especially repeatedly and/or according to a predefined time regime. Preferably, alternatively or cumulatively, the control and computing unit may be further configured to execute the measurement cycle based on a request triggered by a user via an input means. Preferably, the input means may thereby be connected to the control and computing unit in a wireless or wired manner.

Preferably, alternatively or cumulatively, the at least one sensor device (the first sensor device) connected downstream of the dialyser on a dialysis liquid-side and/or, if applicable, a further sensor device connected downstream of the dialyser may be configured to acquire as the at least one signal especially a conductivity of the consumed dialysis liquid in its course over time, further preferably to be configured as a temperature-compensating conductivity probe. Preferably, alternatively or cumulatively, the at least one sensor device (the first sensor device) and/or, if applicable, a further sensor device connected downstream of the dialyser may be configured to acquire as the at least one signal an absorption property of the consumed dialysis liquid in its course over time. Thereby, the absorption property (of a sensor device arranged especially, but not limiting, downstream of the dialyser) especially relates to the absorbance and/or the degree of transmission. Preferably, the absorption property may be acquired at a wavelength or, respectively, in a wavelength range between 200 nm and 320 nm, further preferably between 250 nm and 310 nm and especially at 280+/−15 nm. For this purpose, the sensor device may emit, for example, light with a wavelength in the UV range. Preferably, alternatively or cumulatively, the at least one sensor device (the first sensor device) and/or, if applicable, a further sensor device connected downstream of the dialyser may be arranged in the dialysis liquid outlet line directly at the dialysis liquid outlet, e.g. directly flanged thereto. In all embodiments, it may be especially (independently) preferred that the second sensor device, or, respectively, a further sensor device downstream of the dialyser, is directed at (or, respectively, is configured for acquiring or, respectively, measuring) a different physical and/or chemical variable than the at least one sensor device (the first sensor device) is. The at least one sensor device at or downstream of the dialysis liquid outlet of the dialyser is especially adapted to acquire blood components, especially uremic toxins (e.g. urea, creatinine, uric acid, potassium etc.), that have passed through the filter membrane, in the consumed dialysis liquid flowing out of the dialyser, especially as conductivity probe. In general, it should be noted that the sensor device measures a physical and/or chemical variable that may be proportional to the concentration of a specific substance. The proposed (monitoring) method for recirculation determination or, respectively, measurement does not require actual or, respectively, immediately measured concentration values are.

Preferably, alternatively or cumulatively, the control and computing unit may further be provided and configured to determine a first current recirculation value based on the first signal of the first sensor device and a second current recirculation value based on the second signal of the second sensor device. Thereby, the control and computing unit may further be provided and configured to thereupon make a comparison of the first and second current recirculation values, especially to deduce or, respectively, to determine a, if applicable, present deviation (qualitatively/quantitatively). This offers the advantage that by using the additional information from the at least two different sensor devices or, respectively, measuring probes, especially if based on two different measuring principles such as by means of conductivity versus by means of an optical probe, useful assessments or, respectively, decisions can be made for the monitoring and the safe operation.

For example, the control and computing unit may be further provided and configured to average, based on the comparison of the first and second current recirculation values, preferably an average current recirculation value (as a mean value) of the first and second current recirculation values. This averaging can advantageously improve the reproducibility and serve for suppressing of measurement noise.

Alternatively to an mean value calculation, especially in case of a deviation determinable or, respectively, determined (esp., statistically significant) based on the comparison of the first and second current recirculation values, the control and computing unit may further be provided and configured to determine the (esp., statistically significant) deviation. Further preferably, the control and computing unit may be further provided and configured to perform an (optional) further step based on the determined (esp., statistically significant) deviation of the first and second current recirculation values from each other, especially to automatically make a control decision, preferably in the background or under information to a user via a display means, and/or to query a user via an interactive user interface.

For example, the further step may relate to partially or completely discard the result (i.e. to ignore at least one or both of the first and second current recirculation values). Especially, this may be performed in case of an unquestionable criterion for discarding or, respectively, in the presence of an artifact. Especially the case or, respectively, the artifact may be that the first and/or second current recirculation value is less than 0% or greater than 100%. These values make no sense physically, but may be mathematically possible as artifacts due to insufficient signal quality in the sensor device in question.

For example, alternatively or cumulatively, the further step may relate to a renewed measurement cycle, whereby such a measurement cycle may be initiated automatically and/or suggested or, respectively, triggered to a user via an interactive user interface. A query about the execution of the renewed measurement cycle offers the advantage that the user retains the decision-making authority about whether he considers the initiation of such a measurement cycle as necessary or, respectively, useful at the point in time in question.

For example, the (especially, statistically significant) deviation may be, as a blanket, greater than 1 sigma, further preferably greater than 2 sigma and especially greater than 3 sigma (standard deviation) and/or be, as a blanket, greater than 10 percentage points (in relation to a difference of both the first and second recirculation values measured in %), further preferably greater than or equal to 3 percentage points.

In a further alternatively or cumulatively preferred embodiment, the control and computing unit may be further provided and configured to make an advance decision, based on the comparison of the first and second current recirculation values, with respect to the significance or, respectively, weighting of the deduced deviation and, accordingly, to perform the further step differently.

Thereby, the advance decision may be made based on even further data, especially of state data (e.g. time course: start of treatment versus end of treatment) of the current extracorporeal blood treatment. This serves advantageously the user-convenient consideration of the measurement-physical basic conditions (e.g. at the beginning of treatment with high concentrations of the protagonists/blood substances/toxins sufficient signal quality/optimal measuring window; versus at the end of treatment after already essentially accomplished cleaning/separation of the toxins reduced signal quality/unfavorable signal/noise ratio).

Furthermore, the control and computing unit may be further provided and configured to make the advance decision depending on a relevance range in which the corresponding first or, respectively, second current recirculation value (respectively) falls or, respectively, fall. Thereby, the relevance range may be especially between 0% and 50%, further preferably greater than or equal to 15% recirculation value. Especially, the control and computing unit may be further provided and configured not to discard the measurements if within the relevance range, further preferably not to discard the measurement within the relevance range, but to use it (as valid). In other words, the control and computing unit may decide, even if a deviation of the two compared values is determined from-case-to-case, i.e. depending on the determination whether respectively inside or outside the relevance range, whether the at least one first and/or second current recirculation value lying in the relevance range are to be used or if just not. For example, it may be preferable not to discard a measurement for the first current recirculation value at 15% and a measurement for the second current recirculation value at 12%, but to deduce the reaching of a relevant recirculation. This serves the advantage to be able to inform a user about the result without delay or, respectively, to initiate (automatically and/or by intervention of the user) measures for reduction of the physically occurring recirculation.

Preferably, further at least one third sensor device may be provided on a dialysis liquid-side of the dialyser connected upstream, i.e., a sensor device upstream of the dialyser. Thereby, the third sensor device may be configured to acquire, as the at least one signal, especially a conductivity of the unconsumed dialysis liquid in its course over time. Further preferably, the third sensor device is configured as a temperature-compensating conductivity probe, especially for measuring a concentration of a cation. Alternatively or cumulatively, the third sensor device may be configured (as) an optical sensor. For this purpose, the sensor device may, for example, emit light with a wavelength in the UV range. Preferably, alternatively or cumulatively, the third sensor device and/or, if applicable, a further sensor device connected upstream of the dialyser may be arranged in the dialysis liquid inlet line directly at the dialysis liquid inlet, e.g. directly flanged thereto. In all embodiments it may be especially (independently) preferred that the third sensor device, or, respectively, a further sensor device upstream of the dialyser, is directed at a different physical and/or chemical variable (or, respectively, is configured for acquired or, respectively, measuring such a variable) than the third sensor device is. Especially, it may be thereby preferred that at least one of the sensor devices connected upstream of the dialyser, i.e. the third sensor device and/or the further sensor device upstream of the dialyser, is arranged upstream of the bypass line.

Preferably, alternatively or cumulatively, the third sensor device connected upstream of the dialyser and first sensor device connected downstream of the dialyser may be configured based on one same measurement principle. Thereby, the second sensor device connected downstream of the dialyser is configured based on, in comparison, another measuring principle. Preferably, alternatively or cumulatively, the third sensor device and the first sensor device may be configured to acquire a same physical and/or chemical variable, especially a conductivity, as the respectively corresponding signal, i.e. the third signal of the third sensor device and the first signal of the first sensor device. Thereby, the second sensor device is configured to acquire a physical and/or chemical variable that is different therefrom, i.e. in comparison to the third signal and the first signal, as the corresponding second signal (of the second sensor device).

Preferably, alternatively or cumulatively, the control and computing unit may further be provided and configured to use, in the case of an essentially determinable match of the third signal and the first signal for the base mode, the second signal of the second sensor device in a prioritizing manner for the measurement cycle in order to deduce the presence of a recirculation, especially to determine the current recirculation value.

Preferably, alternatively or cumulatively, a specific embodiment of the extracorporeal blood treatment machine may have the following features: Thereby, the dialyser may comprise a dialysis liquid inlet for fresh dialysis liquid, a dialysis liquid outlet for consumed dialysis liquid and a filter membrane. Thereby, the filter membrane separates a dialysis liquid membrane side, on which the dialyser is connected to a dialysis liquid circuit via a dialysis liquid inlet line and a dialysis liquid outlet line, from a blood membrane side, on which the dialyser is connected or connectable to an extracorporeal blood circuit. Thereby, a bypass line may be provided, by means of which the dialysis liquid side (or, respectively, the dialysis liquid circuit) becomes selectively bridgeable in a bypass mode as the interim module for the interim operation for a respective interim time interval, especially for the first interim time interval as well as for the second interim time interval, in order to temporarily confine dialysis liquid present in the dialyser. For this purpose, at least one check valve electrically connected to the control and computing unit may be arranged between the bypass line and the dialyser at the dialysis liquid inlet line and the dialysis liquid outlet line, respectively. In other words, the respective check valves (controllable by the control and computing unit or, respectively, automatic) serve a bypass circuit or, respectively, side connection circuit.

Thereby, the control and computing unit for controlling the extracorporeal blood treatment machine may be configured or equipable with a storage unit. Alternatively or cumulatively, the control and computing unit may be electrically (or, respectively, signal-wise) connected or connectable via an interface to a storage unit external to the control and computing unit.

The extracorporeal blood treatment machine may further comprise (the following features): A data set (comprised by the extracorporeal blood treatment machine) stored or storable on the storage unit may indicate a number of blood flow rates in the extracorporeal blood circuit, referred to as Q_{B}, suitable for different parameters of the blood treatment machine, and corresponding, preferably analytically pre-determined and/or blanket-set, first and second interim time intervals. Thereby, a respective first interim time interval corresponds to that time duration, within which, in the dialyser at correspondingly set blood flow rate Q_{B} a concentration balancing of the at least one selected or selectable blood component between blood in the extracorporeal blood circuit and dialysis liquid confined in the dialyser has ended exclusively due to diffusion, whereby especially a maximum possible recirculation value is to be assumed or, respectively, assumable. For the determination of the first bolus signal or, respectively, the second bolus signal, the control and computing unit is thus preferably configured to switch the extracorporeal blood treatment machine into the bypass mode for the duration, respectively, of the first interim time interval specified in the data set or, respectively, of the second interim time interval, which is shorter than the first interim time interval. Thereby, the control and computing unit is preferably further configured to operate the extracorporeal blood circuit, preferably simultaneously, at the specified blood flow rate. Thereby, the control and computing unit is preferably further configured, to metrologically acquire, immediately after respective termination of the bypass mode for the first or, respectively, second dialysis liquid bolus respectively created by the bypass mode, in the consumed dialysis liquid draining from the dialyser, by means of the at least one sensor device the signal change corresponding to the respective first or, respectively, second dialysis liquid bolus relative to the base signal as a corresponding first bolus signal or, respectively, second bolus signal.

Optionally, the extracorporeal blood treatment machine may thereby further comprise the calculation model stored on the storage unit. Based on the calculation model, the control and computing unit may be further configured to optionally calculate a current recirculation value taking into account the first bolus signal, the second bolus signal and the base signal. The calculation may thereby preferably take place before the start or at the beginning of a treatment cycle by means of the extracorporeal blood treatment machine. The quantification of the recirculation, if applicable, determined as present in terms of a current recirculation value (in %) supports the precision and thus the benefit for a user. Thus, changes of the treatment parameters or, respectively, settings of the extracorporeal blood treatment machine, especially of the blood flow rate, can be individually optimized or, respectively, adjusted for the respective patient (status). If this is done in advance or at the beginning of the treatment cycle, the adverse recirculation may be avoided in advance, at least to a large extent. This may especially be done iteratively by means of successively repeated measurement cycles, especially automatically, especially in the background of the treatment. In this way, an optimization can be achieved with regard to an efficient, shortest possible treatment duration while at the same time avoiding the disadvantageous recirculation. Furthermore, by using a (quantitatively) determined current recirculation value, it is better possible to distinguish between a still acceptable recirculation with low percentage values, e.g. occurring in case of an incipient shunt blockage or, respectively, shunt throttling, and a (prohibitively) high one. This supports the ability to make an accurate diagnosis what regards the possible cause(s) of the recirculation. Also, based on the comparison of a plurality or, respectively, a history of (quantitatively) determined current recirculation values, observations over several treatment cycles and/or corresponding conclusions and/or predictions in the sense of a maintenance, especially relating to consumables of the blood circuit, can be made.

Optionally, the extracorporeal blood treatment machine may thereby further provide a device for identifying (of) the dialyser and/or for input of the dialyser identification or individual dialyser parameters. This effects the advantage that parameters corresponding to the dialyser, e.g. a theoretical clearance, can be used for a, if applicable, further specification of the calculation model by the storage and calculation unit. This can further increase the significance of the automatically calculated recirculation value for specific constellations or, respectively, diagnoses.

However, as already shown by way of introduction, the present disclosure solves, in view of WO 2020/212 332 A1, the specific and important object of providing a particularly inexpensive, easy-to-perform recirculation measurement, which is not only not dependent on the systemic blood fraction concentration c_{sys}, but is also able to deliver results, especially even quantitatively precise ones, independently of the knowledge of other machine or, respectively, individual therapy parameters or, respectively, dialyser parameters, which are included in the determination in said and the general prior art. Thus, in experiments carried out on the part of the applicant, it could be confirmed that in representative applications the knowledge of the dialyser did not lead to a further improvement of the accuracy or, respectively, reproducibility of the recirculation determination according to the disclosure. As described above, the particular advantages of the present disclosure lie precisely in the fact that an innovative possibility for measurement is provided, which is characterized by reliability or, respectively, reproducibility as well as uniform sensitivity for the total measurement spectrum, while at the same time keeping the technical or, respectively, apparatus-related effort required largely low as well as minimizing the possibility of the unintentional introduction of multiple sources of measurement error.

In this respect, it may alternatively also be preferred to use the present disclosure in a medical or, respectively, clinical situation, in which (or, respectively, whereby) the extracorporeal blood treatment machine just provides no (i.e., not any or, respectively, not necessarily any) device for identification of the dialyser and/or for input of the dialyser identification or individual dialyser parameters. This can be particularly advantageous, for example, in countries with less good medical care and thus represents a contribution to the worldwide availability of modern blood treatment devices with an inexpensive as well as reliable treatment monitoring.

Preferably, alternatively or cumulatively, the storage unit may be firmly integrated in the extracorporeal blood treatment machine. This serves a compact design with simplified assembly in the manufacturing. Furthermore, this protects against manipulation of the manufacturer's contents on the storage unit by third parties.

Preferably, alternatively or cumulatively, the extracorporeal blood treatment machine may further comprise a display device, connected wirelessly or by wire, which is configured for displaying the deduced presence of a recirculation, especially of the determined current recirculation value. Especially, an interface is thereby provided for the connection. For example, the display device may comprise an optical display device such as a, especially interactive, screen and/or an acoustic display device, especially an interactive voice output computer. The display device may thereby preferably be provided dislocally on a, preferably mobile or, respectively, personal, device such as a mobile phone, laptop or the like of the patient and/or of a remotely treating physician and/or be provided as such a device. This effects the advantage of the information and/or of the bio-feedback to a user of the disclosed extracorporeal blood treatment machine such as to the patient and/or the physician or, respectively, the clinical staff. Thus, with the knowledge of the, if applicable, presence of the recirculation or, respectively, of the current recirculation value corresponding changes of the treatment parameters or, respectively, settings of the extracorporeal blood treatment machine, especially of the blood flow rate, can be optimized individually for the respective patient (status) and/or immediately adjusted in real time due to a recirculation that has occurred, for example, only in the course of the dialysis treatment.

Preferably, alternatively or cumulatively, the at least one sensor device and the control and computing unit can be provided in or, respectively, at a separate module device for recirculation determination retrofittable and/or retrofitted to the extracorporeal blood treatment machine. Thereby, the (separate, especially autonomous) module device comprises a sensor unit with at least one sensor device and a connecting section or part. Thereby, the connecting section or part for preferably adaptively connecting (of) the sensor unit to the fluid circuit of the extracorporeal blood treatment machine is provided or, respectively, configured such that the at least one sensor device on the dialysis liquid-side is connected downstream of the dialyser and is configured for acquiring (of) the at least one signal of the physical and/or chemical variable of the consumed dialysis liquid in its course over time. Further, an electronics and evaluation unit of the module device, configured or, respectively, acting as the control and computing unit, operating independently of the extracorporeal blood treatment machine, thereby deduces the presence of the recirculation. Especially, the control and computing unit or, respectively, the electronics and evaluation unit of the module device may thereby determine the current recirculation value. Further, the module device may thereby optionally comprise an input/output interface, preferably combined, which is configured to output to a user the recirculation deduced by the electronics and evaluation unit, especially the determined current recirculation value. Furthermore, the module device may thereby optionally have a power supply configured independently of the extracorporeal blood treatment machine. Thereby, it is equally conceivable that the module device is configured to be (co-)connected to a power supply of the blood treatment machine.

A further, second-mentioned, aspect of the present disclosure relates to a (separate) module device for a determination of a recirculation or, respectively, for a monitoring of a recirculation (rate) in an extracorporeal blood treatment for retrofitting to an extracorporeal blood treatment machine with a dialyser. To avoid repetitions, with respect to essential and preferred technical features of the module device, reference is made to the same features as disclosed with respect to the foregoing first-mentioned aspect of the present disclosure. This module device advantageously serves, for example, to provide an (existing) extracorporeal blood treatment machine, if applicable also of an older type, with an additional functionality.

The module device (for the extracorporeal blood treatment machine and/or of the extracorporeal blood treatment machine) comprises a control and computing unit and a sensor unit electrically connected to the control and computing unit. Thereby, the control and computing unit is configured as an electronics and evaluation unit operating independently of the extracorporeal blood treatment machine. Thereby, the sensor unit has at least one sensor device and a connecting section or part for preferably adaptively connecting the sensor unit to the fluid circuit of the extracorporeal blood treatment machine, such that the at least one sensor device on a dialysis liquid-side is connected downstream of the dialyser. Thereby, the sensor device is configured (and connected/connectable) to measure, for quantitative determination of at least one preferably selected or selectable blood component in the consumed dialysis liquid, at least one signal of a physical and/or chemical variable of the consumed dialysis liquid.

Thereby, the control and computing unit for determination of a presence of a recirculation without blood-side bolus administration is provided and configured therefor:
to acquire the at least one signal measured by the at least one sensor device in its course over time;
to operate the extracorporeal blood treatment in a base mode for continuous operation in which the dialysis liquid flow through the dialyser is admitted,
to measure the signal corresponding to the dialysis liquid consumed in the base mode as a base signal;
to switch the extracorporeal blood treatment from the base mode into an interim mode for interim operation, in which a dialysis liquid amount is confined in the dialyser on the dialysis liquid membrane side, while the blood flow in the extracorporeal blood circuit is operated at a set blood flow rate, whereby the blood on the blood membrane side continues to flow, for the respective duration of a pre-determined and/or predeterminable interim time interval, especially to switch into a bypass mode as the interim mode; subsequently
to change into the base mode, in order to supply the previously confined dialysis liquid amount as a respective dialysis liquid bolus to the at least one sensor device, in order to measure a signal change, corresponding to the dialysis liquid bolus, relative to the base signal as a corresponding bolus signal;
to switch to the interim mode for a first interim time interval for establishment of equilibrium of the mass transfer, whereby a first dialysis liquid amount in the dialyser is confined at least until a concentration equilibrium of the blood component is established on the dialysis liquid membrane side and the blood membrane side of the dialyser, which equilibrium is no longer changing or is only still changing to an unsubstantial degree.

Thereby, the control and computing unit is, for forming a measurement cycle, further provided and configured therefor:
to switch to the interim mode for a second interim time interval, (which is) shorter than the first interim time interval, whereby a second dialysis liquid amount in the dialyser is confined only as long as the concentration equilibrium of the blood component is not yet established,
to deduce, from a deviation of a second bolus signal as result of the second interim time interval compared to a first bolus signal as result of the first interim time interval, the presence of the recirculation.

This enables to display the presently disclosed recirculation determination due to the integration/arrangement of sensors and evaluation/control in the separate (if applicable, mobile) module device, i.e. outside the machine boundary of the primary extracorporeal blood treatment machine, independent of the latter and its technical configuration such as a year/type of construction etc. The retrofittability allows a cost-saving, sustainable upgrade of older blood treatment machines with new functions.

Especially, the module device may have an (optional) input/output interface to a user, which is configured to output to a user the recirculation determined by the control and computing unit. Especially, the module device may have an (optional) power supply configured independently of the extracorporeal blood treatment machine.

Especially the module device may comprise at least two, especially three (optional) check valves for switching into an interim operation of the fluid circuit. In an extended configuration further preferred over the aforementioned core equipment of the module device, the module device further optionally has a bypass check valve in the bypass line, i.e. for fluid communication of the dialysis liquid source with the dialysis liquid sink, as well as at least the (first) check valve (i.e. input valve to the dialyser), connected upstream of the dialyser, in the dialysis liquid inlet line, especially both check valves (i.e., the first check valve upstream/before the dialyser and the second check valve downstream/after the dialyser), in order to switch to an interim operation, especially to the bypass mode, of the fluid circuit. In other words, the check valves of the extracorporeal blood treatment machine, which are required for the bypass circuit described above, are associated for retrofitting in such an optional embodiment of the extended module device or, respectively, provided at/in this, especially duplicated to those of the blood treatment machine. Thus, even older blood treatment machines can be equipped with the disclosed new functionality without retooling or, respectively, software updates, therefore without any restrictions. Furthermore, the module device serves a further reduction of the complexity or, respectively, improvement of the human-machine interaction. Especially, there is no need for a user to start the bypass mode on the blood treatment machine.

A further, third-mentioned, aspect of the present disclosure relates to a method for monitoring a recirculation (rate) in an extracorporeal blood treatment using an extracorporeal blood treatment machine according to the first-mentioned aspect of the present disclosure and/or using a module device for recirculation determination according to the second-mentioned aspect of the present disclosure. To avoid repetitions, reference is made to the disclosure especially to the first-mentioned aspect with respect to essential and preferred technical features. Thereby, the method for monitoring comprises the following (monitoring) steps: An optional step relates to an identification of the dialyser. In an initial step a predetermining of the first interim time interval takes place, within which a concentration equilibrium of a preferably previously selected blood component, preferably urea, is established. Especially, the predetermining of the first interim time interval may be done in dependence of a set blood flow rate Q_{B} or blanketly. In a further initial step a predetermining of the second interim time interval, within which the concentration equilibrium of the blood component is not yet established, takes place. Consequently, the second interim time interval relates to a shorter duration than the first interim time interval.

Thereafter, a step of deducing a first bolus signal as result of the first interim time interval and a step of deducing a second bolus signal as result of the second interim time interval take place in any order.

The step of deducing the first bolus signal has the following (especially, sequential) substeps: i) operating the extracorporeal blood treatment machine in the base mode and measuring the signal corresponding to the dialysis liquid consumed in the base mode as a base signal; ii) switching over from the base mode into the interim mode, especially into the bypass mode, for the duration of the first interim time interval; and subsequently to substep (ii) or, respectively, to the termination thereof (iii) changing (back) into the base mode to supply the previously confined dialysis liquid amount as the first dialysis liquid bolus to the at least one sensor device, and subsequent measuring of the first bolus signal. Thereby, the first bolus signal relates to a signal change (or, respectively, a response) corresponding to the first dialysis liquid bolus relative to (or, respectively, compared to) the base signal. While the substeps (ii) and (iii) must be performed in subsequent sequence or, respectively, are (automatically) executed by the control and computing unit configured for this purpose, it is possible to execute substep (i) temporally before or after the substeps (ii) with (iii) in combination.

Before or after the step of the deducing of the first bolus signal the step of the deducing of a second bolus signal as result of the second interim time interval with corresponding substeps takes place. Thereby, the corresponding substeps are analogous to those of the step of the deducing of the first bolus signal.

Based on the deduced second bolus signal in relation (or, respectively, in comparison) to the deduced first bolus signal, essential to the disclosure, a step of deducing the presence of the recirculation from (or, respectively, based on the determination of) a deviation of the second bolus signal compared to the first bolus signal takes place. Especially, the step of the deducing of the presence of the recirculation may thereby be performed by way of (or, respectively, as) calculating a current recirculation value (R in %) by using a calculation model according to a ratio, whereby the ratio is defined as the second bolus signal in relation to the first bolus signal. Thereby, the skilled person understands that the ratio can also be used in the form of a reciprocal value or otherwise modified characteristic value of the aforementioned definition, which he knows to take into account mathematically accordingly in the sense of the evaluation. The recirculation or, respectively, the current recirculation value may be, in an optional step of a display, displayed or, respectively, output qualitatively, preferably (semi-)quantitatively, to a user.

Advantageously, an actually present recirculation may thus be determined without having to determine the concentration of a blood component in the blood of the patient's body in advance by separate blood sampling, and consequently a fully automated, time-saving and safe blood treatment method can be enabled.

### BRIEF DESCRIPTION OF THE DRAWINGS

The disclosure is described in more detail below according to preferred embodiments with reference to the attached drawing. Showing:

FIG. 1 a preferred embodiment of a dialysis machine as an extracorporeal blood treatment machine according to the disclosure, illustrating an (optional) module device;

FIG. 2 a first embodiment of a time course, according to the disclosure, of a signal measured at a sensor device of dialysis machine according to the disclosure in temporal switching sequence of a short bypass mode followed by a long bypass mode, illustrating the case of 0% recirculation, whereby the sensor device is arranged directly behind the dialysis liquid outlet of the dialyser;

FIG. 3 a second embodiment of a time course, according to the disclosure, of the measured signal, illustrating the case of 25% recirculation, whereby there are otherwise comparable conditions compared to the first embodiment in FIG. 2;

FIG. 4 a third embodiment of a time course, according to the disclosure, of the signal measured at a sensor device of dialysis machine according to the disclosure, in temporal switching sequence of a long bypass mode followed by a short bypass mode, changed with respect to the first embodiment in FIG. 2 or, respectively, with respect to the second embodiment in FIG. 3, for illustration of the fundamental independence of the switching sequence, insofar as related to the respectively corresponding temporal course section of the long bypass mode versus that of the short bypass mode.

The figures are of a schematic nature only and are intended solely for the understanding of the disclosure. The same elements are denoted with the same reference signs.

### DETAILED DESCRIPTION

Embodiments of the present disclosure are described below on the basis of the corresponding Figures.

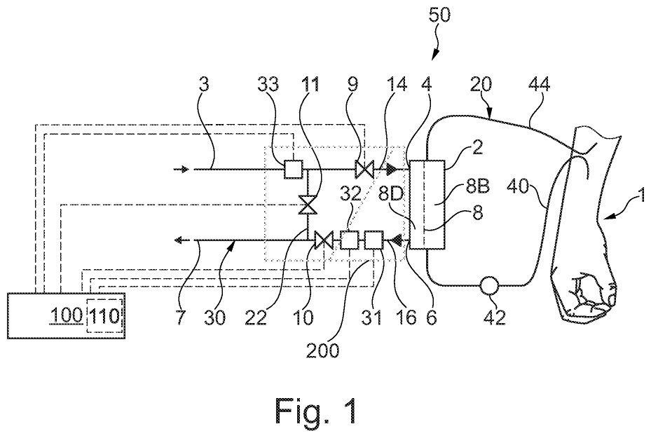

FIG. 1 schematically shows a preferred embodiment of a disclosed extracorporeal blood treatment machine for determination of the recirculation, configured as a dialysis machine 50 with a dialyser 2 comprising a dialysis liquid inlet 4 and a dialysis liquid outlet 6. Furthermore, FIG. 1 shows an (optional) module device 200, as further detailed below.

Blood is withdrawn from a patient 1 via an arterial blood line 40 by means of a blood pump 42 and directed to the dialyser 2. There it is purified and then returned to the patient 1 via the venous line 44 as an extracorporeal blood circuit 20.

The dialyser 2 is, for the purpose of blood purification, further equipped with a semi-permeable filter membrane 8, which separates a (dialysate-side) dialysis liquid membrane side 8D from a (blood-side) blood membrane side 8B or, respectively, divides the volume of the dialyser into these two partial volumes. Thereby, the dialysis liquid membrane side 8D is in fluid communication with a dialysis liquid inlet line 14 and a dialysis liquid outlet line 16 of a dialysis liquid circuit 30. The dialysis liquid circuit 30 thereby represents a flow path between a dialysis liquid source 3 towards a dialysis liquid sink 7. The blood membrane side 8B is in fluid communication with the extracorporeal blood circuit 20.

For example, the dialyser 2 may be configured in form of a hollow fiber module (operable in countercurrent-flow, cocurrent-flow and/or cross-flow), whereby the filter membrane 8 is configured in form of hollow fiber bundles around which the dialysis liquid (or, respectively, the dialysate) of the dialysis liquid circuit 30 flows. Thereby, the dialysis liquid inlet line 14 is connected to the dialysis liquid inlet 4; and the dialysis liquid outlet line 16 is connected to the dialysis liquid outlet 6 of the dialyser 2.

In the dialysis liquid circuit 30, a bypass line 22 is further provided, with which the dialyser 2 or, respectively, the dialysis liquid membrane side 8D of the dialyser 2 can be bypassed, i.e., fluid-dynamically circumvented.

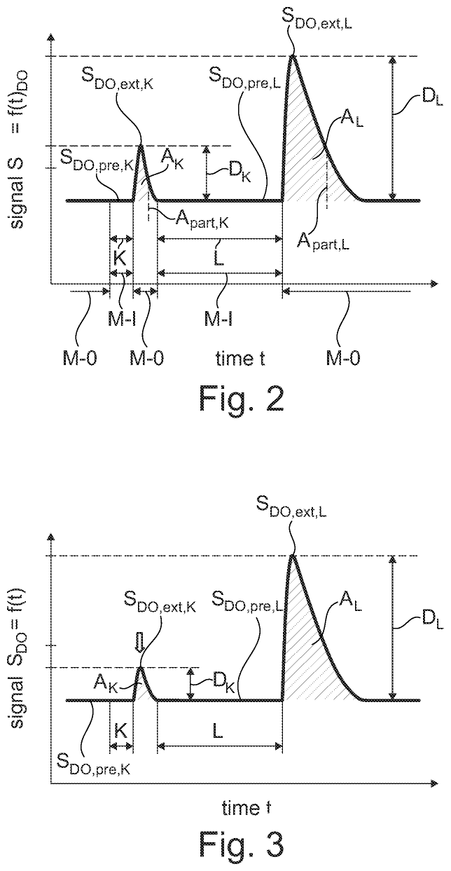

This takes place (cf. FIGS. 2 to 4) in a switched (over) bypass mode M-I as interim mode. Thereby, it is switched (over) into the bypass mode M-I to temporally replace a base mode M-0 of a continuous dialysis operation [i.e., of a dialysis operation in which the dialysis liquid is continuously conveyed (across) or, respectively, flows across the or, respectively, along the dialysis liquid membrane side 8D] or, respectively, to change from the base mode M-0 into the bypass mode M-I (temporarily or, respectively, in an interim manner). For this purpose, a check valve 9, 10 is provided in the dialysis liquid inlet line 14 and the dialysis liquid outlet line 16, respectively, by means of which the dialysis liquid inlet line 14 and the dialysis liquid outlet line 16 can be opened or, respectively, closed and thus either, with the opened check valves 9, 10, open the flow path for fresh dialysis liquid through the dialysis liquid inlet line 14, the dialysis liquid membrane side 8D of the dialyser 2 and the dialysis liquid outlet line 16, or close this flow path by closing at least the check valve 9, ideally both check valves 9, 10. In other words, if these check valves 9, 10 are closed, the dialysis liquid flows past the dialyser 2 through the bypass line 22 when the bypass check valve 11 is open. This state of operation defines the bypass mode M-I (FIG. 2).

In the preferred embodiment of the dialysis machine 50 according to the disclosure, a first sensor device 31 is located in the dialysis liquid outlet line 16 or, respectively, downstream of the dialysis liquid outlet 6 (and, for example, but not limitingly, upstream of the check valve 10) as the at least one sensor device and further an optional second sensor device 32.

Furthermore, it can be seen from FIG. 1 that an optional third sensor device 33 may be provided in the dialysis liquid inlet line 14 or, respectively, upstream of the dialysis liquid inlets 4 (and e.g., but not limiting, upstream of the check valve 9).

The respective sensor devices 31, 32, 33 each measure a chemical and/or physical variable of the dialysate. The first sensor device 31 and the third sensor device 33 are preferably sensor devices for conductivity determination, especially temperature-compensating conductivity probes.

The optional second sensor device 32 is configured as optical sensor device, for example. For this purpose, the second sensor device 32 takes measurements in the dialysis liquid flowing past using of UV/VIS spectroscopy depending on the absorption range of the specific blood component to be measured at the wavelength that can be absorbed by this blood component, in order to preferably enable a concentration determination of this blood component or a corresponding parameter determination. Concentration/parameter determinations by absorption measurement are generally known and are therefore not explained in detail here.

In addition to the measurement methods specified above, alternative measurement methods are also conceivable. The respective sensor device, especially the first, second and/or third sensor device 31, 32 and 33, may be positioned in the flow direction, before or, respectively, upstream (as exemplarily shown in FIG. 1) or after or, respectively, downstream of the bypass line 22.

The measured signal values generated at the first sensor device 31 and at the optional second and third sensor device 32, 33 are transmitted to a control and computing unit 100 and evaluated by it.

The control and computing unit 100 is in mutual information exchange with at least the check valves 9, 10 and the sensor devices 31, 32, 33, i.e. it receives and sends information, respectively, from the check valves 9, 10 and the sensor devices 31, 32, 33 or, respectively, to the check valves 9, 10 and the sensor devices 31, 32, 33. Especially with respect to the respective sensor devices 31, 32, 33, the information relates to a time-varying signal S_{DO}=ƒ(t) [cf. ordinate of FIGS. 2 to 4], which is each based on the measurement of a chemical and/or physical variable of the dialysate for measuring (of) the blood component to be measured.

Further, the control and computing unit 100 is connected to or equipped with a storage unit 110. On this storage unit 110 at least one data set is stored or, respectively, storable, with which the dialysis machine 50 can be controlled depending on the specified or, respectively, received information.

The dialysis machine 50 further has a further check valve in form of the bypass check valve 11 in the bypass line 22, which is also in information exchange contact with the control and computing unit 100 and can be controlled by the latter, i.e. opened and closed. The bypass check valve 11 is provided to open or to close the flow path for fresh dialysis liquid via the bypass line 22. The fresh dialysis liquid is obtained from a dialysis liquid supply unit as the dialysis liquid source 3.

Downstream of the dialysis liquid source 3 there may be (optionally for any embodiments) a balance chamber (not shown) which is configured to balance fresh dialysis liquid flowing into and consumed dialysis liquid flowing out of the dialysis liquid circuit 30. Thereby, the balance chamber (not shown) may preferably be fluid-dynamically arranged such that it is located between the dialysis liquid source 3 and an opening point of the dialysis liquid inlet line 14 into the bypass line 22 and between the dialysis liquid sink 7 and an opening point of the bypass line 22 into the dialysis liquid outlet line 16.

On the blood membrane side 8B of the dialyser 2, the extracorporeal blood circuit 20 is connected to the dialyser 2. The blood circuit 20 has at least one arterial blood line 40, which connects an arterial patient inlet to a blood-side dialyser inlet and to which a blood pump 42 is arranged, and a venous blood line 44, which connects a blood-side dialyser outlet to a venous patient inlet. The blood pump 42 is also in (mutual) information exchange contact with the control and computing unit 100 and is controlled by it.

As further schematically illustrated by the grey dotted lines in FIG. 1, the (optional) module device 200 may be provided separately. Thereby, the (separate) module device 200 may be retrofitted or, respectively, retrofittable to the dialysis machine 50 as the extracorporeal blood treatment machine. In a core configuration of the optional module device 200 (as shown in FIG. 1 to the right of the gray dotted slanted line), the module device 200 comprises an electronics and evaluation unit for recirculation deduction configured as the control and computing unit 100, operating independently of the dialysis machine 50, as well as a sensor unit electrically connected to the control and computing unit 100 and comprising at least one (first) sensor device 31. Thereby, the sensor unit has, in addition to the at least one (first) sensor device 31, a connecting section or part for preferably adaptively connecting (of) the sensor unit to the dialysis liquid circuit 30 of the dialysis machine 50, such that the at least one sensor device 31 (if applicable, 32) on the dialysis liquid-side is connected downstream of the dialyser 2 and is used for acquiring (of) the signal S_{DO}. The module device 200 is, via the dialysis liquid inlet line 14 upstream of the dialysis liquid membrane side 8D, fluid-connected or, respectively, fluid-connectable thereto. The module device 200 is, via the dialysis liquid outlet line 14 downstream of the dialysis liquid membrane side 8D, fluid-connected or, respectively, fluid-connectable thereto.

In a preferred configuration extended beyond the aforementioned core equipment of the module device 200 (as shown in FIG. 1 to the left of the gray dotted slanted line), the module device 200 further optionally has a bypass check valve 11 in the bypass line 22 for fluid communication of the dialysis liquid source 3 with the dialysis liquid sink 7 and at least the (first) check valve 9 connected upstream of the dialyser 2 (i.e. inlet valve to the dialyser 2) in the dialysis liquid inlet line 14, especially both check valves 9, 10 (i.e. the first check valve 9 upstream/before the dialyser 2 and the second check valve 10 downstream/after the dialyser 2), in order to switch to an interim operation, especially the bypass mode, of the fluid circuit. In other words, the check valves of the dialysis machine 50, which are required for the bypass circuit described above, belong in such an optional embodiment to the retrofitted/retrofittable module device 200. Thereby, these are at least two check valves 9, 11 (i.e. for the interim mode), especially three check valves 9, 10, 11 (i.e. for the bypass mode M-I). A third check valve is the bypass check valve 11, which allows the dialysis liquid to flow around the dialyser 2 in the bypass mode when the check valves 9, 10 are closed. In this way, the dialysis liquid bypasses the dialyser 2 in the bypass mode. In general (i.e., independently of an optional module device 200), it should be noted that the second check valve 10 does not necessarily have to be closed. For example, it may be preferred from-case-to-case that with the second check valve 10 open it may be further ultrafiltrated, i.e. liquid withdrawn from the patient's blood. The longer the bypass mode lasts, the more advantageous it is not to close the second check valve 10. However, in the case of a short bypass mode and/or low ultrafiltration rates, it is better for the performance of the saturation process if, in addition to the first check valve 9, also the second check valve 10 has been/is closed for the bypass mode.

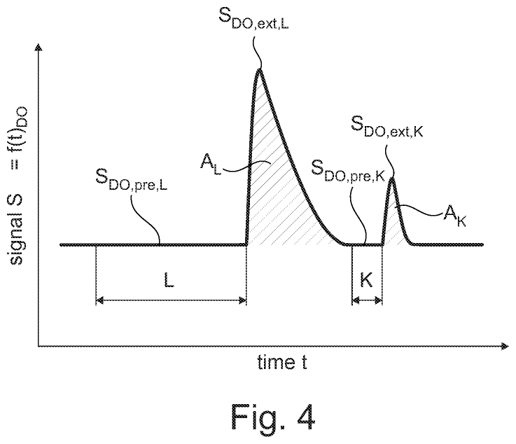

In the operation of the dialysis machine 50 (FIG. 1; with reference to all embodiments of the time courses of the measured signals shown in FIGS. 2 to 4), a patient 1 is first connected to the extracorporeal blood circuit 20. Subsequently, the control and computing unit 100 retrieves from the data set which is stored on the storage unit 110 a target blood flow rate Q_{B} and respective target time values for the respective duration of a manufacturer-pre-determined respective interim time interval for the bypass mode. With reference to FIG. 2 (and FIGS. 3, 4), the pre-determined interim time intervals for the bypass mode M-I relate to a first interim time interval L for establishment of equilibrium of the mass transfer and a second interim time interval K, shortened in relation thereto, with respect to a kinetic point of time in advance of the establishment of equilibrium (according to the first interim time interval L). Optionally, patient-dependent minimum and maximum upper limits for the target blood flow rate Q_{B} can be thereby taken into account in the data set. The time value for the first interim time interval L in the bypass mode M-I is thereby preferably selected/set such that during the bypass mode M-I a (near) diffusion equilibrium is achieved in the dialyser 2 for the signal-relevant blood component to be used for the at least one sensor device 31, 32 even under the worst circumstance, namely a maximum possible or maximum expected recirculation (e.g. recirculation value of 20% to 30%). The rule here is that the lower the blood flow rate Q_{B} and the larger the dialyser, the longer it takes to reach (near) diffusion equilibrium. Depending on the specific blood component to be acquired for the signal, which is optionally selected from several possible blood components before the treatment, the outputted first interim time interval L may additionally be larger or smaller.

After the control and computing unit 100 has retrieved the first interim time interval L and the second interim time interval K as the target time values and the target blood flow rate Q_{B} or, respectively, these have been entered manually, it sets the blood pump 42 to the target blood flow rate Q_{B}. When the target blood flow rate Q_{B} is reached and a pre-determined value for the dialysis liquid flow through the dialysis liquid circuit 30 is also reached, a steady state of continuous operation may be assumed or, respectively, has been established. The steady state of the continuous operation, especially with respect to the continuous flow-through and continuously occurring interfacial physical mass transfer or, respectively, dialysis processes, defines a base mode M-0 (see FIG. 2).

Now, in the base mode M-0 for the continuous operation, in which the dialysis liquid flow through the dialyser 2 is admitted, the signal corresponding to the consumed dialysis liquid can be measured by the first sensor device 31 as the at least one sensor device and/or by the second sensor device 32 as a (leveled or, respectively, constant) base signal S_{DO, pre}.

Respectively after the (reaching of) the base mode M-0, the dialysis machine 50 is switched into the bypass mode M-I by the control and computing unit 100 (see especially FIG. 2, further FIGS. 3, 4). This means that for the respective pre-determined interim time intervals for the bypass mode M-I, on the one hand for the first interim time interval L (equilibrium) and on the other hand for the second interim time interval K (disequilibrium; driving concentration gradient non-negligible)), namely in any order, the check valves 9, 10 are closed, i.e. the dialysis fluid located between the check valves 9, 10 is confined, and the bypass check valve 11 in the bypass line 22 is open, such that the flow path of the fresh dialysis liquid from the dialysis liquid source 3 leads via the bypass line 22 to the dialysis liquid sink 7.

For the duration of the bypass mode, the blood pump 42 is now operated at the set blood flow rate while the dialysis liquid is stationary on the dialysis liquid membrane side 8D. As a result, the blood component that correlatively influences or, respectively, determines the signal S_{DO} according to the measurement of the chemical and/or physical variable of the dialysate via its concentration, passes from the blood flowing through the blood membrane side 8B of the dialyser 2 via the filter membrane 8 into the dialysis liquid stationary on the dialysis liquid membrane side 8D of the dialyser 2.

In the first interim time interval L, the blood component correlatively influencing or, respectively, determining the signal S_{DO} via its concentration accumulates in the stationary first dialysis liquid amount until a diffusion equilibrium is (essentially) reached on the blood membrane side 8B and the dialysis liquid membrane side 8D. Since the blood in the extracorporeal blood circuit 20 continuously continues to flow, the diffusion equilibrium concentration of the blood component determining the signal S_{DO} in the first dialysis liquid amount corresponds (at the latest) at the end of the first interim time interval L essentially to the concentration of the blood component determining the signal S_{DO} present in the blood of the extracorporeal blood circuit 20. The latter in turn corresponds at this point of time to the concentration of the blood component determining the signal S_{DO} present in the blood of the patient's body. Consequently, the diffusion equilibrium concentration of the blood component determining the signal S_{DO} in the stationary first dialysis liquid amount corresponds to the concentration of the blood component determining the signal S_{DO}) present in the blood of the patient's body.

In contrast, in the comparably (significantly) shorter or, respectively, “short”, second interim time interval K, a second dialysis liquid amount is confined in the dialyser 2 only until the concentration equilibrium of the blood component determining the signal S_{DO} is not yet established.

Both processes are independent of each other, but proceed under otherwise constant conditions however along the identical mass transfer, or, respectively, diffusion kinetics. Only the processes are terminated at different times, according to the respective pre-determined interim time interval, for the purpose of the measurement at the at least one sensor device 31, 32.

Also during the second interim time interval K, the blood is still pumped through the dialyser 2 by the blood pump 42. Due to the closed check valves 9 and 10, the dialysate volume 8D is confined. Thereby, it gradually adopts the concentration of substances dissolved in the blood and permeable, e.g. uric acid, creatinine, sodium, potassium, etc. The second interim time interval K as the duration of the “short” bypass circuit is selected such that a complete equalization between the blood membrane side 8B and the dialysis liquid membrane side 8D does not take place. In laboratory tests, it has been shown that for the second interim time interval K as duration of the “short” bypass circuit less than or equal to 1 minute is sufficient. Preferably the duration is 10 to 20 seconds, especially (ca.) 14 seconds.

If a recirculation is present, then the blood entering into the dialyser 2 during the (“short”) second interim time interval K, i.e. during this time in advance of the establishment of equilibrium, is influenced by the returning venous blood and thus recirculation-affected.

Accordingly, it is essential to the present disclosure (irrespective of other technical features) that the second dialysis liquid amount of the dialysis liquid membrane side 8D confined during the (“short”) second interim time interval K, thus also the second dialysis liquid bolus, in (or, respectively, in the case of) the presence of a recirculation, is recirculation-affected.

In contrast, during the (“long”) first interim time interval L, as described above, the confined first dialysis liquid amount of the dialysis liquid membrane side 8D fully adapts to the blood that is continuously conveyed through the dialyser 2. Since the arterial blood is no longer influenced in its composition by the dialysis liquid, the venous blood flowing back does not lead to any further change of the arterial blood, even in the presence of a recirculation (i.e., in the case of recirculation being present, despite of this).

Therefore, after a sufficiently long time in the bypass mode or, respectively, at the latest at the end of the first interim time interval L, the blood in the arterial line 40 is no longer recirculation-affected.

Accordingly, it is essential for the present disclosure (irrespective of other technical features) that the first dialysis liquid amount of the dialysis liquid membrane side 8D enclosed or, respectively, confined during the (“long”) first interim time interval L, consequently also the first dialysis liquid bolus, is not recirculation-affected even in (or, respectively, in the case of the) presence of a recirculation.

When the respective pre-determined interim time interval for the bypass mode M-I, namely on the one hand for the first interim time interval L and on the other hand for the second interim time interval K, has been reached, the control and computing unit 100 cancels the bypass mode M-I. Consequently, it closes the bypass check valve 11 in the bypass line 22 and simultaneously opens the valves 9, 10 in the dialysis liquid inlet line 14 and the dialysis liquid outlet line 16 in order to return into the base mode M-0 or, respectively, to switch (back). Now in the base mode M-0, fresh dialysis liquid flows again from the dialysis liquid source 3 through the dialysis liquid membrane side 8D of the dialyser 2.

The respective first or second dialysis fluid amount (or, respectively, the associated delta volume) that has previously, i.e. in the respective first or, respectively, second interim time interval, been stationary or, respectively, confined on the dialysis fluid membrane side 8D, consequently flows in form of a respective first or, respectively, second dialysis fluid bolus through the dialysis liquid outlet line 16 in the direction of the dialysis liquid sink 7, whereby it passes the first sensor device 31 and the second sensor device 32. The latter measures, cf. FIGS. 2 to 4, as a result of the enrichment of the blood component correlatively influencing or, respectively, determining the signal S_{DO} via its concentration or, respectively, a respectively corresponding signal deflection or, respectively, a signal response S_{DO}=ƒ(t), referred to as corresponding bolus signal.

After termination of the second interim time interval K as duration of the “short” bypass circuit or, respectively, side connection circuit in the bypass mode M-I, it is again switched back into the base mode M-0 or, respectively, the main connection circuit, i.e., the check valves 9 and 10 open and the bypass check valve 11 closes. Fresh dialysis liquid flows into the dialyser 2 and displaces the previously at least partially saturated second dialysis liquid amount in form of the second dialysis liquid bolus. When the second dialysis liquid bolus passes the first sensor device 31 and/or the second sensor device 32, a momentary signal deflection is thus seen there as the second bolus signal.

As can be further seen from FIGS. 2 to 4, the bolus signal corresponding to the first or, respectively, second interim time interval L, K is thereby recorded or, respectively, acquired in form of a peak or, respectively, extremum S_{DO, ext, L}, S_{DO, ext, K} (e.g. conductivity and/or light absorption peak representing the concentration of the blood component) and/or in form of a total area integral A_{L}, A_{K} or, respectively, partial area integral A_{L, part}, A_{K, part} (hatched marked portions) running underneath.

As can be further seen from FIGS. 2 to 4, the value at the peak or, respectively, in the extremum of the signal is denoted as S_{DO, ext, K} or, respectively, S_{DO, ext, L}. As shown above, the value for the first dialysis liquid bolus in the extremum S_{DO, ext, L} corresponds to the blood-side value without recirculation (i.e. at ca. 0% recirculation).

In contrast, the steady state value shortly before the start or end of the respective first or, respectively, second bypass circuit in the bypass mode M-I is referred to as S_{DO,pre,L} or, respectively, S_{DO, pre, K}.

If the signal under consideration is the absorbance (e.g. second sensor device 32 in FIG. 1), then a maximum is always to be expected as the extremum. If, on the other hand, the signal is the conductivity (e.g. first sensor device 31 in FIG. 1) or sodium concentration (or the concentration of any other blood component that occurs both in the blood and in the dialysis liquid), a minimum may also occur.

In the possible event that no significant deflection occurs, in that direction and magnitude of the deflection depends on the gradient between dialysate membrane side 8D and blood membrane side 8B, the dialysis machine may preferably (alternatively or cumulatively to further technical features) be configured to briefly increase the gradient prior to performing the recirculation determination or, respectively, the measurement cycle by changing the composition and thus the conductivity of the dialysis liquid. In order to minimize any undesired sodium withdrawal or, respectively, addition by adjusting the dialysis liquid, the conductivity of the dialysis liquid may subsequently be set back to the original value.

If the control and computing unit 100 registers a (statistically significant) deviation of the second bolus signal S_{DO, ext, K}; AK; A_{part, K}, occurring as result of the second interim time interval K, compared to a first bolus signal S_{DO, ext, L}; AL; A_{part, L}, occurring as result of the first interim time interval L, it deduces the presence of a recirculation.

FIGS. 2 to 4 show (with reference to the schematic illustration of FIG. 1) a respective time course of the signal S_{DO}=ƒ(t) of the measurement recording (especially conductivity of the dialysis liquid of the blood component to be acquired for the signal) at the at least one sensor device 31, 32 (especially at the first sensor device 31) of a dialysis machine 50. Thereby, the at least one sensor device 31, 32 is arranged in flow direction before or, respectively, upstream (as exemplarily shown in FIG. 1) or after or, respectively, downstream of the check valve 10 of the dialysis liquid outlet line 16. Furthermore, the at least one sensor device 31, 32 is thereby arranged in the flow direction upstream or, respectively, upstream (as exemplarily shown in FIG. 1) or after or, respectively, downstream of the opening point of the bypass line 22 into the dialysis liquid outlet line 16. The temporal offset of the peak due to a displacement of the sensor device 31, 32 in the downstream direction is disregarded as negligible for the sake of simplicity. However, due to the above consideration, it may be preferred that the at least one sensor device 31, 32 (especially the first sensor device 31, preferably as temperature-compensating conductivity probe) is arranged directly at the dialysis liquid outlet 6 (downstream), especially flanged thereto.

In the diagrams of FIGS. 2 to 4, the double arrows L or, respectively, K parallel to the time axis designate the first interim time interval L and the second interim time interval K as the respective time periods in which the bypass mode M-I (denoted only in FIG. 2, analogously in FIGS. 3, 4) is active, i.e. at the at least one sensor device 31, 32 no dialysis liquid flowing from the dialysis liquid membrane side 8D flows past.

Before and/or during the bypass mode M-I or, respectively, L, K, the at least one sensor device 31, 32 thus measures a (steady state) constant signal as the base signal S_{DO, pre} in the dialysis liquid for the (specific) blood component. Thereby, the (largely) constant nature results from the fact that the first or, respectively, second dialysis liquid amount (or, respectively, the corresponding first or, respectively, second delta volume), in which (or, respectively, in which) the blood component, correlatively influencing the signal S_{DO} via its concentration, accumulates due to diffusion, is confined between the check valves 9, 10 and fresh dialysis liquid from the dialysis liquid source 3 does not flow around the sensor device. The acquired base signal S_{DO, pre} can be considered as correlating to the concentration of the blood component influencing or, respectively, determining the signal S_{DO} via its concentration, at the dialyser outlet under normal, known treatment/operation conditions, i.e., in the base mode M-0.

The value S_{DO, pre, L} corresponds to the steady state value, i.e. the base signal S_{DO, pre}, shortly before start or end of the first interim time interval L, especially before start or end of the longer lasting bypass circuit. It may also correspond to the steady state value, i.e. the base signal S_{DO, pre}, shortly before start or end of the second interim time interval K, especially before start or end of the shorter lasting bypass circuit, denoted as S_{DO, pre, K}, or vice versa.

After termination of the bypass mode M-I or, respectively, L, K, the at least one sensor device 31, 32 measures a distinct respective second peak in the time course of the signal S_{DO}=ƒ(t), since after opening of the check valve 10 the previously respectively confined first or, respectively, second dialysis liquid amount now flows past at the at least one sensor device 31, 32 as the first or, respectively, second dialysis liquid bolus.

Once the first or, respectively, second dialysis liquid bolus has completely passed the sensor device 31, 32, the signal corresponds again to the base signal S_{DO, pre}.

The control and computing unit (with reference sign 100 in FIG. 1) calculates, on the one hand, a second difference, denoted as D_{K} (FIGS. 2, 3), from the maximum peak of the second bolus signal S_{DO, ext, K} and the base signal S_{DO, pre} as well as a first difference, denoted D_{L} (FIGS. 2, 3), from the maximum peak of the first bolus signal S_{DO, ext, L} and the base signal S_{DO, pre}. The following two equations describe this:

$${D}_{L} = {S}_{DO , ext , L} - {S}_{DO , pre , L}$$

$${D}_{K} = {S}_{DO , ext , K} - {S}_{DO , pre , K}$$

If the second difference D_{K} and the first difference D_{L} differ from each other, the control and computing unit (with reference sign 100 in FIG. 1) consequently determines the presence of the recirculation or, respectively, derives this qualitative conclusion from it or, respectively, detects the recirculation as such.

Preferably, the control and computing unit 100 may quantitatively determine a current recirculation value R (in %) based on the calculation model predefined and retrievable from the storage unit 110 (FIG. 1). For this purpose, alternatively or cumulatively, three different ratios may be used:

The ratio of the second difference to the first difference is called the delta ratio V_{D}:

$${V}_{D} = \frac{{D}_{K}}{{D}_{L}}$$

Thus, the current recirculation value R can be determined as a function of this ratio or, respectively, delta ratio V_{D}:

R=ƒ(V_{D})

Alternatively or cumulatively for evaluation of the signal differences, the respective total areas (hatched portions) below the two signal deflections A_{K} and A_{L} and/or respective partial areas (indicated by dashed vertical dividing line) below the two signal deflections A_{part, K} und A_{part, L} may also be brought into relation:

$${V}_{A} = \frac{{A}_{K}}{{A}_{L}}$$

$$total area integral ratio$$

Or, respectively,

$${V}_{A , part} = \frac{{A}_{part , K}}{{A}_{part , L}}$$

$$partial area integral ratio$$

In this way, the recirculation can also be determined as function of the area ratios in terms of the total area integral ratio or, respectively, partial area integral ratio:

R=ƒ(V_{A})

R=ƒ(V_{d,part})

As the comparison of FIGS. 2 and 3 illustrates, it has been shown that the height of the peak or, respectively, the areal expression of the second bolus signal S_{DO, ext, K} in relation to the height of the peak or, respectively, to the areal expression of the first bolus signal S_{DO, ext, L} decreases with increasing recirculation.

In other words, a respective one of the above ratios decreases with increase of the recirculation or, respectively, of the current recirculation value R. In other words, this means an indirect correlation.

Thus, including at least one of the above ratios, i.e. of the delta ratio V_{D} and/or of the total area integral ratio V_{A} and/or of the partial area integral ratio V_{A, part}, a current recirculation value R can be quantitatively determined or, respectively, calculated using the (pre-determined) calculation model.

Thus, FIG. 2 exemplarily shows the case of 0% for the current recirculation value R; and FIG. 3 shows the case of 25% for the current recirculation value R. From the comparison of the otherwise comparable situations or, respectively, illustrations, it can be seen that the signal deflection/peak S_{DO, ext, K} or, respectively, the area integral A_{K} below the signal deflection after termination of the “short” bypass circuit or, respectively, of the second interim time interval K in FIG. 3 becomes smaller or, respectively, is reduced in comparison to the corresponding case in FIG. 2 (indicated in FIG. 3 by the downward pointing symbol arrow above the second bolus signal S_{DO, ext, K}).

In FIG. 4, otherwise analogous to FIG. 2, it is illustrated that the sequence of the respective interim time intervals, in which the bypass mode M-I (indicated in FIG. 2 only, analogous in FIG. 4) is active, may also be selected in reverse, such that first a “long” bypass circuit is performed, followed by a “short” one, corresponding to the first interim time interval L followed by the second interim time interval K. This may be advantageous especially at the beginning of an extracorporeal blood treatment. Insofar, when putting the patient on, the bypass is activated anyway, so that this time interval can already be used to saturate the dialysate-side volume 8D completely up to establishment of equilibrium, i.e. in the first time interval L. As soon as the first bolus signal at the dialysate outlet could be determined after switching over into the base mode M-0 (or, respectively, into the main connection) and the signal has subsequently levelled to the base signal S_{DO, pre}, the “short” bypass circuit may be carried out over the second time interval K.

As mentioned above, for performing (of) the method for monitoring the dialysis machine 50 according to the disclosure at least one sensor device, especially the first sensor device 31 and/or the second sensor device 32, is provided. If both sensor devices 31, 32 should be used at the same time, a plausibility check of the deduced recirculation may be performed by comparing the recirculation determined by the first sensor device 31, preferably calculated as current recirculation value R, and the recirculation determined by the second sensor device 32, preferably calculated as current recirculation value R, with each other. If both current recirculation values R differ, e.g. by more than 10 percentage points (difference) or by more than 1 sigma, the deduced recirculation may be discarded and a renewed measurement cycle based on the first and the second interim time interval L, K be started. On the other hand, it may also be preferred not to discard, in a measurement for the first current recirculation value at 15% and in a measurement for the second current recirculation value at 12%, these measurements, but to deduce the reaching of a relevant recirculation. For example, a traffic light color “yellow” may then indicate an incipient critical recirculation via an (interactive) user interface.

Alternatively, both current recirculation values R of the first and of the second sensor devices 31, 32 can be averaged.

Furthermore, it is conceivable to select the underlying at least one sensor device, i.e. the first sensor device 31 or the second sensor device 32, depending on the gradient corresponding to the chemical and/or physical variable between the dialysis liquid inlet (i.e. the dialysis liquid inlet 4 and/or the dialysis liquid inlet line 14) and the dialysate outlet (i.e. the dialysis liquid outlet 6 and/or the dialysis liquid outlet line 16). If, for example, the third sensor device 33 for the dialysis liquid inlet and the first sensor device 31 for the dialysis liquid outlet measure approximately the same conductivity value, the corresponding gradient is thus almost (or, respectively, quasi) zero, so that no significant signal deflections would be detected. Especially in this case of a reduced probe, but not limiting, it may be preferred to prioritize a recirculation determination using the second sensor device 32 (e.g. by optical measurement).

Of course, it is also possible to incorporate the respective signals of both sensor devices, i.e. of the first sensor device 31 or, respectively, of the second sensor device 32, individually from the outset into an equation or, respectively, into a calculation model for recirculation determination that takes both signals into account.

1. An extracorporeal blood treatment machine comprising:
a dialyser;
a control and computing unit; and
at least one sensor device connected on a dialysis liquid-side downstream of the dialyser and electrically connected to the control and computing unit,
the control and computing unit being configured for determination of a presence of a recirculation without blood-side bolus administration and further configured:
to acquire at least one signal of a physical and/or chemical variable of a consumed dialysis liquid over time, being measured by the at least one sensor device for quantitative determination of at least one blood component in the consumed dialysis liquid,
to operate the extracorporeal blood treatment machine in a base mode for continuous operation in which the dialysis liquid flow through the dialyser is admitted,
to measure the at least one signal corresponding to the consumed dialysis liquid in the base mode as a base signal;
to switch the extracorporeal blood treatment machine from the base mode into an interim mode for interim operation, in which a dialysis liquid amount is confined in the dialyser on a dialysis liquid membrane side, while blood flow in a extracorporeal blood circuit is operated at a set blood flow rate, whereby blood on the blood membrane side continues to flow, for a respective duration of a pre-determined and/or predeterminable interim time interval;
to change into the base mode, in order to supply a previously confined dialysis liquid amount as a dialysis liquid bolus to the at least one sensor device, in order to measure a signal change, corresponding to the dialysis liquid bolus, relative to the base signal as a bolus signal; and
to switch to the interim mode for a first interim time interval for establishment of equilibrium of the mass transfer, whereby a first dialysis liquid amount in the dialyser is confined at least until a concentration equilibrium of the at least one blood component is established on the dialysis liquid membrane side and the blood membrane side of the dialyser, which equilibrium is no longer changing or is only still changing to an unsubstantial degree,
the control and computing unit is, for forming a measurement cycle, further configured:
to switch to the interim mode for a second interim time interval shorter than the first interim time interval, whereby a second dialysis liquid amount in the dialyser is confined only as long as the concentration equilibrium of the blood component is not yet established, and
to deduce a presence of the recirculation from a deviation of a second bolus signal as a result of the second interim time interval compared to a first bolus signal as a result of the first interim time interval.

2. The extracorporeal blood treatment machine according to claim 1, wherein:
the first bolus signal and second bolus signal respectively correspond to a maximum peak in a signal of the at least one sensor device immediately after a release of the respective first or, respectively, second dialysis liquid bolus from the dialyser;
the control and computing unit is further provided and configured therefor:
to deduce a delta ratio from a second difference, calculated from the maximum peak of the second bolus signal and the base signal, in relation to a first difference, calculated from the maximum peak of the first bolus signal and the base signal; and
to determine, based on the deduced delta ratio and a predefined calculation model, a current recirculation value.

3. The extracorporeal blood treatment machine according to claim 1, wherein:
the first bolus signal and the second bolus signal respectively correspond to a total area integral, adjusted by the base signal, in the signal of the at least one sensor device immediately after the release of the respective first or, respectively, second dialysis liquid bolus from the dialyser, and
the control and computing unit is further and configured:
to deduce a total area integral ratio from a second total area integral in relation to a first total area integral; and
to determine a current recirculation value based on the total area integral ratio and a predefined calculation model.

4. The extracorporeal blood treatment machine according to claim 1, wherein:
the first bolus signal and the second bolus signal respectively correspond to a partial area integral, adjusted by the base signal, in the signal of the at least one sensor device immediately after the release of the respective first or, respectively, second dialysis liquid bolus from the dialyser, and
the control and computing unit is further provided and configured:
to deduce a partial area integral ratio from the second partial area integral in relation to the first partial area integral; and
to determine a current recirculation value, based on the partial area integral ratio and a predefined calculation model.

5. The extracorporeal blood treatment machine according to claim 2, wherein the predetermined calculation model for calculation of the current recirculation value considers a dialysis liquid flow rate of the dialysis liquid circuit, a blood flow rate in the extracorporeal blood tubing system of the blood circuit, and a theoretical clearance of the dialyser.

6. The extracorporeal blood treatment machine according to claim 1, wherein the at least one sensor device comprises a first sensor device connected downstream of the dialyser on a dialysis liquid-side and a second sensor device connected downstream of the dialyser,
the second sensor device directed to a physical and/or chemical variable different from the first sensor device,
the first sensor device and the second sensor device:
being configured to acquire, as the at least one signal, an absorbance property, and/or
being arranged in the dialysis liquid outlet line directly at the dialysis liquid outlet.

7. The extracorporeal blood treatment machine according to claim 6, wherein:
the control and computing unit is further configured:
to determine a first current recirculation value based on the first signal of the first sensor device and a second current recirculation value based on the second signal of the second sensor device.

8. The extracorporeal blood treatment machine according to claim 1, further comprising at least one third sensor device connected upstream of the dialyser on a dialysis liquid-side, the at least one third sensor device:
is configured to acquire, as the at least one signal an absorbance property, and/or
is arranged in the dialysis liquid inlet line directly at the dialysis liquid inlet; and/or
is arranged upstream of the bypass line.

9. The extracorporeal blood treatment machine according to claim 8, wherein:
the at least one third sensor device and the at least one first sensor device are configured based on a same measuring principle to acquire a same physical and/or chemical variable as the respectively corresponding third signal of the at least one third sensor device and the first signal of the at least one first sensor device; and
the second sensor device is configured based on a different measurement principle to acquire a different physical and/or chemical variable than the corresponding second signal of the second sensor device;
the control and computing unit being further configured:
in the case of a match of the third signal and the first signal, essentially determinable for the base mode, to use the second signal of the second sensor device in a prioritizing manner for the measurement cycle in order to deduce the presence of a recirculation.

10. The extracorporeal blood treatment machine according to claim 1, wherein:
the dialyser comprises a dialysis liquid inlet for fresh dialysis liquid, a dialysis liquid outlet for consumed dialysis liquid and a filter membrane separating a dialysis liquid membrane side, on which the dialyser is connected to a dialysis liquid circuit via a dialysis liquid inlet line and a dialysis liquid outlet line, from a blood membrane side, on which the dialyser is connected or connectable to an extracorporeal blood circuit;
a bypass line is provided by which the dialysis liquid membrane side is selectively bridgeable in a bypass mode as the interim mode for the interim operation for the first interim time interval as well as for the second interim time interval, in order to temporarily confine dialysis liquid present in the dialyser, for which purpose at least respectively one check valve electrically connected to the control and computing unit is arranged at the dialysis liquid inlet line and the dialysis liquid outlet line between the bypass line and the dialyser;
the control and computing unit is configured or equipable with a storage unit and/or is electrically connected or connectable via an interface with a storage unit external with respect to the control and computing unit; and
the extracorporeal blood treatment machine further comprises:
a data set stored or storable on the storage unit, which indicates a number of blood flow rates in the extracorporeal blood circuit suitable for different parameters of the blood treatment machine and corresponding first interim time intervals, within which, in the dialyser at correspondingly set blood flow rate under the assumption of a maximum possible recirculation value, a concentration balancing of the at least one selected or selectable blood component between blood in the extracorporeal blood circuit and dialysis liquid confined in the dialyser has ended exclusively due to diffusion,
such that the control and computing unit, for determination of the first bolus signal or, respectively, the second bolus signal, switches the extracorporeal blood treatment machine to the bypass mode respectively for the duration of the first interim time interval indicated in the data set or, respectively, of the second interim time interval, which is shorter than the first interim time interval, and operates the extracorporeal blood circuit at the indicated blood flow rate and acquires, immediately after respective termination of the bypass mode for the respective first or, respectively, second dialysis liquid bolus produced by the bypass mode, in the consumed dialysis liquid draining from the dialyser by the at least one sensor device, the signal change corresponding to the respective first dialysis liquid bolus or, respectively, second dialysis liquid bolus relative to the base signal as a corresponding first bolus signal or, respectively, second bolus signal.

11. The extracorporeal blood treatment machine according to claim 1, wherein the storage unit is firmly integrated in the extracorporeal blood treatment machine.

12. The extracorporeal blood treatment machine according to claim 1, wherein the extracorporeal blood treatment machine further comprises a display device, connected wirelessly or by wire, which is configured for displaying the deduced presence of a recirculation.

13. The extracorporeal blood treatment machine according to claim 1, wherein the at least one sensor device and the control and computing unit are provided externally in or, respectively, at a separate module device for recirculation determination, which is retrofittable and/or retrofitted to the extracorporeal blood treatment machine, whereby the module device comprises:
a sensor unit with:
the at least one sensor device;
a connecting section or part; and
an electronics and evaluation unit configured as the control and computing unit, operating independently of the extracorporeal blood treatment machine, which deduces the presence of the recirculation.

14. A module device for a determination of a recirculation for retrofitting an extracorporeal blood treatment machine with a dialyser, the module device comprising:
a control and computing unit which is configured as an electronics and evaluation unit operating independently of the extracorporeal blood treatment machine; and
a sensor unit, electrically connected to the control and computing unit, with:
at least one sensor device; and
a connecting section or part,
wherein the control and computing unit is configured to determine a presence of a recirculation without blood-side bolus administration, and is further configured:
to acquire the at least one signal, being measured by the at least one sensor device,
to operate the extracorporeal blood treatment in a base mode for continuous operation in which the dialysis liquid flow through the dialyser is admitted,
to measure the signal corresponding to the dialysis liquid consumed in the base mode as a base signal,
to switch the extracorporeal blood treatment from the base mode into an interim mode for interim operation, in which a dialysis liquid amount is confined in the dialyser on the dialysis liquid membrane side, while the blood flow in the extracorporeal blood circuit is operated at a set blood flow rate, whereby the blood on the blood membrane side continues to flow, for the respective duration of a pre-determined and/or predeterminable interim time interval,
to change into the base mode, in order to supply the previously confined dialysis liquid amount as a respective dialysis liquid bolus to the at least one sensor device, in order to measure a signal change, corresponding to the dialysis liquid bolus, relative to the base signal as a corresponding bolus signal,
to switch to the interim mode for a first interim time interval for establishment of equilibrium of the mass transfer, whereby a first dialysis liquid amount in the dialyser is confined at least until a concentration equilibrium of the blood component is established on the dialysis liquid membrane side and the blood membrane side of the dialyser, which equilibrium is no longer changing or is only still changing to an unsubstantial degree, and
wherein:
the control and computing unit is, for forming a measurement cycle, further provided and configured:
to switch to the interim mode for a second interim time interval shorter than the first interim time interval, whereby a second dialysis liquid amount in the dialyser is confined only as long as the concentration equilibrium of the blood component is not yet established,
to deduce, from a deviation of a second bolus signal as result of the second interim time interval compared to a first bolus signal as result of the first interim time interval, the presence of the recirculation.

15. A method for monitoring a recirculation in an extracorporeal blood treatment comprising the steps of:
predetermining a first interim time interval within which a concentration equilibrium of a blood component is established;
predetermining a second interim time interval, which is shorter than the first interim time interval, within which the concentration equilibrium of the blood component is not yet established;
deducing a first bolus signal as result of the first interim time interval comprising the following substeps:
operating an extracorporeal blood treatment machine in a base mode while measuring the signal corresponding to the consumed dialysis liquid as a base signal;
switching over from the base mode into an interim mode, for a duration of the first interim time interval;
changing back into the base mode to supply the previously confined dialysis liquid amount as the first dialysis liquid bolus to the at least one sensor device, and subsequently measuring the first bolus signal as a signal change, corresponding to the first dialysis liquid bolus, relative to the base signal;
deducing a second bolus signal as result of a second interim time interval, before or after the step of deducing of the first bolus signal, the step of deducing the second bolus signal comprising the following substeps:
operating the extracorporeal blood treatment machine in the base mode;
measuring the signal corresponding to the dialysis liquid consumed in the base mode as a base signal;
switching over from the base mode into the interim mode for the duration of the second interim time interval;
changing back into the base mode to supply the previously confined dialysis liquid amount as the second dialysis liquid bolus to the at least one sensor device, and subsequently measuring of the second bolus signal as a signal change, corresponding to the second dialysis liquid bolus, relative to the base signal; and
deducing the presence of the recirculation from a deviation of the second bolus signal compared to the first bolus signal.
